(19) United States
(12) Patent Application Publication
(10) Pub. No.: US 2010/0021555 A1
(43) Pub. Date: Jan. 28, 2010

(54) Compositions containing high omega-3 and low saturated fatty acid levels

(76) Inventors: Karl Geiringer, Waitakere City (NZ); Karen Manson, Surrey (GB); Garth James Smith Cooper, Auckland (NZ)

Correspondence Address: DUANE MORRIS LLP - San Diego, 101 WEST BROADWAY, SUITE 900, SAN DIEGO, CA, 92101-8285, US

(21) Appl. No.: 11/665,445

(22) PCT Filed: Oct. 14, 2005
(86) PCT No.: PCT/IB05/04078

§ 371 Date: Sep. 28, 2009

(30) Foreign Application Priority Data
Oct. 15, 2004 (NZ) NZ 535964

(51) Int. Cl. A61K 35/20; C12P 7/64; A61K 31/202; C12N 1/00; A61P 25/18; A61P 25/28; A61P 3/10; A61P 35/00; A23L 1/30; A23K 1/16

(52) U.S. Cl. 424/535; 435/134; 514/560; 435/243; 426/601; 426/2

(57) ABSTRACT

Food products and supplements comprising the essential unsaturated fatty acids EPA or DHA at high concentrations relative to other n-3 HUFAs and with relatively low levels of saturated fatty acids (myristic and palmitic acids) are provided. These compositions can be made in commercial quantities in a cost effective manner by culture of selected microorganisms. The food products and supplements are suited to human health needs which are unable to be met from fish oil origins. Also provided are EPA containing food products obtained from animals.

### FIELD

The field includes novel compositions for use in an animal or human diet, including compositions having controlled amounts of saturated and unsaturated fatty acid components. The field also includes novel methods of making the compositions, and methods of treatment of a subject using the compositions provided herein.

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art.

Dietary intakes of myristic (C14:0) and/or palmitic (C16:0) acids have been associated with an increased risk of developing cardiovascular disease (CVD) breast cancer and insulin resistance (see World Health Organisation and Food and Agricululture Organisation Joint Expert Committeee Report Diet, Nutrition and the Prevention of Chronic Diseases, WHO Technical Report Series 916, WHO Geneva (2003)). On the other hand, dietary intake of the omega-3 highly unsaturated fatty acids (n-3 HUFAs) DHA and EPA has been associated with a decreased risk of developing cardiovascular disease and with other beneficial health effects. Omega-3-fatty acids in general are known to be beneficial in reducing the incidence of coronary heart disease (Lands, Fish and Human Health Academic Press (1986)). Dietary intake of compositions containing substantially only one of either EPA or DHA in their n-3 HUFA fraction (hereinafter referred to as EPAOIL(s) and DHAOIL(s) respectively) may also be beneficial to the health of people in different life stages, nutritional, and disease states.

While highly purified n-3 preparations are available for pharmaceutical use, it may seem surprising that little, if any, attention has been given to developing process for the manufacture of compositions that comprise EPAOIL or DHAOIL, and at the same time contain relatively low levels of C14:0 and C16:0. In fact, in some cases recent developments appear to point the art further toward reliance on compositions containing relatively high levels of these potentially harmful fatty acids.

Even within a desirable target for humans of less than 10% of daily energy intake from saturated fatty acids, recommendations have been made that intake of foods rich in C14:0 and C16:0 should be replaced by intake of food with a lower content of these particular fatty acids in lipid. At this level of saturated fatty acid intake, a person consuming an total daily diet providing 13 megajoules MJ) of energy and obtaining all their saturated fatty acids from a food source relatively rich in C16:0 and C14:0 (e.g. milk) might obtain 3.25 grams of C14:0 and up to 10 grams of C16:0 (providing approximately 1% and 3% of total energy intake respectively). The U.S. Food and Drug Administration (FDA) has advised that supplements of EPA and DHA of up to 2 grams a day may be safe. Small planktivorous pelagic fish body oil (SPPFBO) is an industrial standard for Northern Hemisphere fish oil used in human dietary supplementation, SPPFBO has status approved by the FDA (FDA GRAS Notice No GRN 000102) and contains 18% EPA, 12% DHA, 15.5% C16:0 and 6.5% C14:0. Thus at safe levels of supplementation n-3HUFA supplements including fish oil supplements can provide sufficient C14:0 and C16:0 to alter dietary load of these potentially detrimental fatty acids significantly.

Dietary deficiency studies in rats in the 1920s demonstrated that n-3 fatty acids are essential (i.e. can not be produced de novo by mammals). The possibility that the population intake of these might be associated with the incidence of atherosclerosis had been presented by the 1950s. (Lancet, 270 (6919) 381-3 (1956)) Epidemiological studies in the 1960s and 70s began to provide evidence of an association between dietary intake of n-3 HUFAs and the incidence of other disease states including inflammatory and autoimmune disorders at a population level. (Bang et al., Lancet (1971) 1; 1143-5 Hirai et al., Lancet (1980)). 2;1 132-3

In the 1980s and 1990s, widely publicised results of human dietary supplementation trials using compositions containing both EPA and DHA (from fish oil) led to the increased consumption of dietary supplements and the registration of at least two n-3 HUFA-based pharmaceuticals in Europe. At the same time, human supplementation trials began to suggest that supplementation with EPAOILs and DHAOILs had divergent health effects. For example, the results of human dietary supplementation trials with highly purified EPA suggest that EPAOILs may be useful for treating certain neurodegenerative and neuropsychiatric disorders (see U.S. Pat. No. 6,689,812 and U.S. Pat. No. 6,384,077). Pure EPA has been approved for pharmaceutical use to treat cardiovascular disease in Japan since 1990. Also human dietary supplementation with DHA-rich oils (produced from micro-algae) suggest that DHAOILs may be especially important in maternal and infant nutrition. (See Uauy R. et al., in J Pediatr 143 (4 Suppl) S1-8 (2003)).

In most countries, dietary guidelines and safety recommendations do not discriminate between EPA and DHA. For example, the U.S. National Institutes of Health Dietary Reference Intake recommends a minimum intake of EPA and DHA combined corresponding to 0.06-0.12% of energy consumed each day. For a 13 megajoule diet this equates to approximately between 200 mg and 400 mg of EPA and DHA per day.

Certain fish are a known source of omega-3-fatty acids, including DHA. (see U.S. Pat. No. 4,670,285). While many fish oils provide significant levels of C14:0 and C16:0, it is unfortunately often the case that these fish oils are unusable for human consumption because of contamination with environmental pollutants that are toxic (e.g. heavy metals and PCB's). Fish oils may also have a fishy odor and taste that is unpleasant, thus rendering them unsatisfactory for use in edible compositions. The cost of purifying fish oils for food and general dietary supplement use to the point where substantially only one of either EPA or DHA remains together with relatively low levels of C14:0 and C16:0 is prohibitive.

Marine microorganisms also are known to contain omega-3-fatty acids. In particular, various species of dinoflagellates are known to contain and DHA. Harrington et al., “The Polyunsaturated Fatty Acids of Marine Dinoflagellates” J. Protozoal, 17:213-219 (1970), characterize the fatty acid content of eight photosynthetic and one heterotrophic marine dinoflagellate, and conclude that the dinoflagellates are a primary producer group of docosahexaenoic acid and contribute substantial amounts of that compound to the marine food chain. Successful cultivation of dinoflagellates to produce an edible oil containing DHA and EPA is very problematic. Dinoflagellates in general are very slow growing and are shear sensitive. Guillard et al., Dinoflagellates, Academic Press (1984).

In order for a culture of microorganisms to be useful for production of lipids for food use the productivity of yield of total fatty acids needs to be sufficiently high so as to allow for cost effective production to be carried out. A particular problem with devising culture techniques to grow biomass with high yields of EPAOILS is that an inverse correlation has been observed between the EPA content of a microorganism and its growth rate. This is partly related to the fact that lower temperature conditions are conducive to EPA yield, while higher temperatures are associated with higher growth rates. Consequently, the need to reduce time-dependent costs (which increase as growth rate declines) forces a practical trade off between EPA content and growth rate in order to obtain the highest yield of EPAOIL for the lowest cost. Existing approaches to producing EPA from microalgal biomass have resulted in microbial biomasses containing less than 5% EPA as a proportion of dry weight (Wen, Z. and Chen F., Process Biochemistry 38: 523-529 (2002)) in comparison to those approaches to producing DHA-rich microorganisms which have resulted in microbial biomasses containing more than 20% DHA a proportion of dry weight and which can be calculated as an equivalent or lower potential process cost (see U.S. Pat. No. 6,566,123). Additionally, those culture conditions that lead to the highest EPA productivity in many cases have also been shown to favor accumulation of C14:0 and or C16:0 in microalgal lipids. Thus, better methods of culturing microbial biomasses to produce higher levels of DHA and in particular EPA and also having relatively low levels of C14:0 and/or C16:0.or C14:0 and/or C14:0 to EPA ratios are needed.

In summary, the problems with current approaches for the manufacture of compositions that comprise EPAOILs and DHAOILs (those containing substantially only one of either EPA or DHA in their n-3 HUFA fraction) are undesirable because they i) contain undesirably high levels of C14:0 and/or C16:0; ii) are produced with such low lipid productivities as to be prohibitive on the basis of cost for general food and dietary supplement application, or iii) are otherwise not suitable for general food and dietary supplement application (e.g. which have unpleasant taste or odor associated with fish oils). There is need for EPAOILs and DHAOILs which are free from these problems, which have relatively low C14:0 and C16:0 levels, that can be manufactured efficiently and which are affordable.

### BRIEF SUMMARY

The inventions described herein meet this need. The inventions described and claimed herein have many attributes and embodiments including, but not limited to, those set forth or described or referenced in this Summary and elsewhere. The inventions are not limited to or by the features or embodiments identified in this Summary, which is included for purposes of illustration only and not restriction.

In a first aspect, edible compositions are provided that contain desired amounts of n-3 HUFA fatty acids (e.g. EPA or DHA) in combination with relatively low levels of C16:0 and C14:0. The compositions are useful as nutritional and dietary supplements, which may contain, for example, nutrients specific to human requirements at different life stages and in different nutritional states, health conditions, and/or disease states. In certain embodiments, the compositions are formulated as nutritional supplements that are manufactured by heterotrophic or primarily heterotrophic production of micro-algae. In other embodiments, alternative methods of producing the compositions are used. Certain embodiments of compositions (e.g. nutritional supplements) comprise between about 18% and about 50% by weight EPA, less than about 11% myristic acid, and less than about 20% palmitic acid where the ratio of EPA to DHA in the composition is at least about 6:1. Alternatively, the ratio of EPA to DHA in the composition is at least about 7:1. to 9:1. In a related embodiment the ratio of EPA to all the total percentage of all other n-3 HUFAs in the composition is at least about 7:1. to 9:1. The compositions can be in the form of a nutritional supplement. Such compositions may be formulated to be suitable for non-aquaculture animal feed supplement, for example where composition is manufactured by a process involving heterotrophic or largely heterotrophic production of micro-algae.

Certain compositions are prepared by at least one process from biomass material of microalgal origin to produce an oil extract containing no more than 50% EPA and relatively low levels of C14:0 and C16:0. Certain preferred embodiments of compositions (e.g. nutritional supplements) comprise at least about 18% by weight EPA; less than about 20% palmitic acid (C16:0); less than about 11% myristic acid (C14:0); and at least about 0.1% alpha linolenic acid (18:3 n-3), wherein the ratio of EPA to DHA is at least about 6:1, and where the composition is made by a primarily heterotropic culture of cultivated micro organism.

In other preferred embodiments, the compositions comprise at least about 18% by weight EPA; less than about 20% palmitic acid (C16:0); less than about 11% myristic acid (C 14:0); wherein the ratio of EPA to DHA is at least about 6:1, and where the composition is made by a primarily heterotropic culture of cultivated microorganism whereby the total fatty acids productivity is at least about 5 mg per liter in a batch culture or 1 mg per liter per day in a continuous culture.

Thus, in some embodiments, compositions, including nutritional supplements are produced by culture of a commercially cultivated micro-organisms (including microalgae). In other preferred embodiments compositions, including nutritional supplements are produced by culture of a commercially cultivated plants or commercially reared animals. Some compositions described herein are made from a cultured microalga, including but not limited to any microalga capable of being grown in a heterotrophic or primarily heterotrophic process. A preferred nutritional supplement is made by a heterotrophic production of microorganisms, specifically by a heterotrophic production of microalgae. A suitable cultured microalga is a heterotrophically (e.g. solely heterotropic) or mixotrophically (primarily heterotropicly) grown culture of the micro-alga Nitzschia laevis (UTEX 2047). Also provided are nutritional supplements produced by a commercially cultivated microorganism, plant, or commercially reared animal where specified amounts of n-3 HUFA fatty acids, myristic acid, and palmitic acid are obtained without the a need for further purification or dilution after the growth of the organism is terminated.

In a preferred embodiment, the compositions are formulated as a nutritional for human consumption, including as non-limiting examples as one or more of food ingredients, functional foods, dietary supplements and nutraceuticals. Thus, nutritional and dietary supplement products for direct human consumption are also provided. Suitable compositions may include whole cells, fatty acid esters, amides, triglycerides, phospholipids, any combination of which is formulated into a powder, soft gel, is microencapsulated, and preferably is combined with an excipient or antioxidant to provide a product suitable for oral administration. Certain compounds, compositions, and cells provided herein are combined with an excipient or antioxidant to provide a product suitable for oral administration.

In other embodiments, one or more food products harvested from non-aquaculture food producing animals that have been fed with compositions described herein (e.g. in a form suitable for oral administration). Suitable food products include milk and dairy products derived from milk, meat, offals, and eggs. Also provided are infant formulas. Such food products are, in a preferred aspect, provided in a form suitable for oral administration to non-aquaculture food producing animals with due regard to the type of animal; for instance provision in extruded form or microencapsulated or in a form protected against attack by intra-ruminal organisms.

EPA-rich foods are provided, for example where the foods are produced via supplementation of the diets of non-aquaculture food producing animals with compositions comprising substantially only EPA as their n-3 HUFA and relatively low or non-detectable levels of C14:0 and C16:0.

Preferably ingestion of such foods or supplements produce a measurable increase in the level of EPA in serum and red blood cells in animals over time with no significant concomitant increase in C14:0 or C16:0.

In another aspect, nutritional and dietary supplements for direct human use are provided. In one embodiment these are produced by supplementing the diet of non-aquaculture food producing animals with the composition rich in n-3 HUFA fatty acids (e.g. EPA) as previously described in this section, and harvesting the food product and/or tissue exhibiting a modified lipid profile from the food-producing animal.

Also provided are compositions for oral administration that produce a measurable increase in the level of EPA in serum and red blood cells of a human subject over a specified time period with no significant concomitant increase in C14:0 or C16:0 relative to that which might be observed via the consumption of other known preparations containing mixtures of n-3 HUFAs (including those with substantially only one n-3HUFA or only one n-3HUFA).

Certain compositions provided herein are useful to prevent, ameliorate, or treat certain conditions or disorders including but not limited to those selected from diabetes (type I, and type II), glycaemic disorders diabetes-associated hypertension, cancer, osteoarthritis, autoimmune diseases, rheumatoid arthritis, inflammatory and auto-immune diseases other than arthritis, respiratory diseases, neurological disorders, neurodegenerative disorders, renal and urinary tract disorders, cardiovascular disorders, cerebrovascular disorders, degenerative diseases of the eye, psychiatric disorders, reproductive disorders, visceral disorders, muscular disorders, metabolic disorders, prostatic hypertrophy and prostatitis, impotence and male infertility, mastalgia, male pattern baldness, osteoporosis, dermatological disorders, dyslexia and other learning disabilities, cancer cachexia, obesity, ulcerative colitis, Crohn's disease, anorexia nervosa, burns, osteoarthritis, osteoporosis, attention deficit/hyperactivity disorder, and early stages of colorectal cancer, lung and kidney diseases, and disorders associated with abnormal growth and development.

Ingestion by humans of foods and nutritional supplements provided herein will preferably produce a measurable increase in the level of EPA in serum and red blood cells over time with no significant concomitant increase in C14:0 or C16:0 relative to consumption of other known preparations mixtures of n-3 HUFAs (including those with substantially only one n-3HUFA or only one n-3HUFA).

Ingestion by humans of foods and nutritional supplements provided herein will, in certain preferred embodiments, produce a measurable increase in the level of other beneficial fatty acids such as DPA, DHA and CLA in serum and red blood cells or other tissues over time with no significant concomitant increase in C14:0 or C16:0.

These and other aspects and embodiments of the inventions described and claimed herein will be apparent from and throughout the application and claims, all of which shall be considered to be a part of the written description thereof.

### BRIEF DESCRIPTION OF THE DRAWINGS

FIG. 1 is a graph showing expression of EPA in milk in a feeding trial (see Example 3)

FIG. 2 is a graph showing expression of DPA in milk in a feeding trial (see Example 3).

FIG. 3 is a graph showing expression of DHA in milk in a feeding trial (see Example 3)

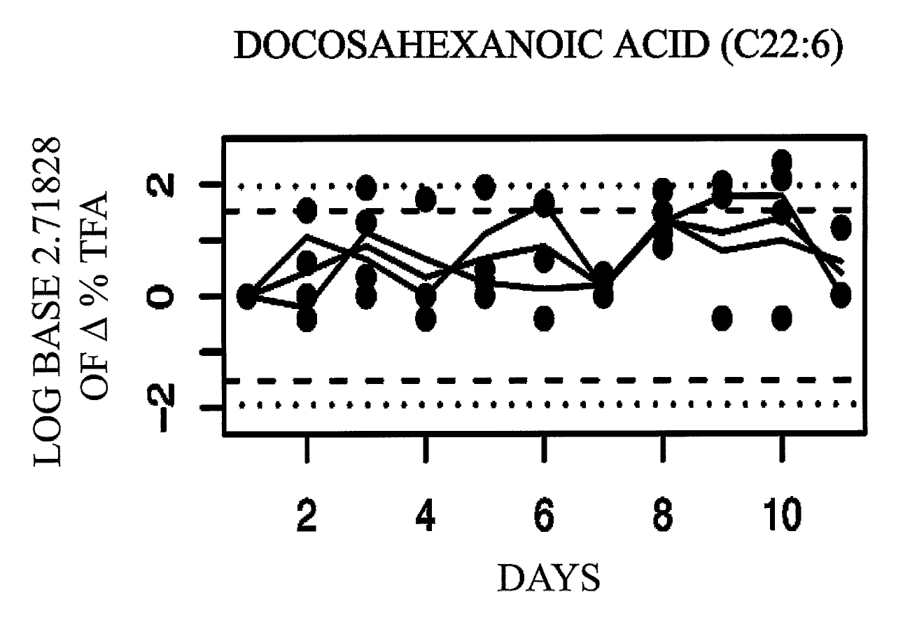

FIG. 4 is a graph showing expression of C16:0 in milk in a feeding trial (see Example 3).

FIG. 5 is a showing expression of CLA in milk in a feeding trial (see Example 3).

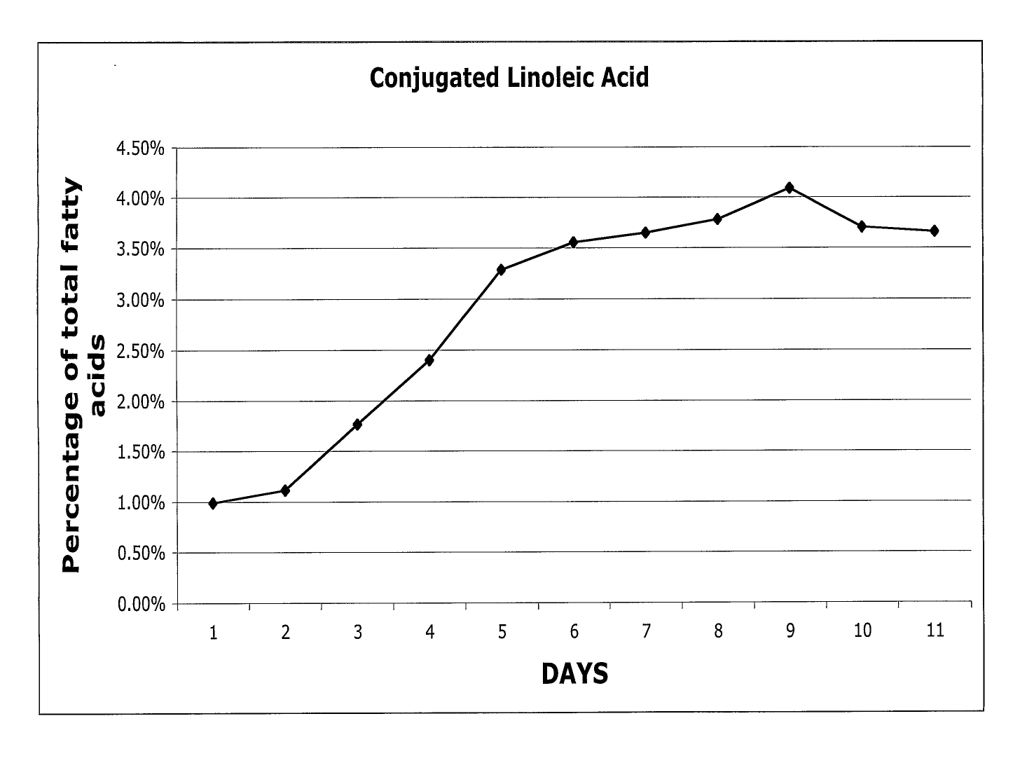

### DESCRIPTION

### Definitions

“Fatty acid (FA)” refers to a chemical compound having a backbone of carbon atoms, the bonds between some of which may be “unsaturated”, with an acid (COOH) moiety at an alpha end of the backbone in the case of a “free” acid. The other end is the omega (w) end. For the purpose of this specification, the term does not exclude salts and esters (including but not limited to ethyl or cholesterol esters, amides, phospholipids, or mono, di- or tri-glycerides) thereof.

The term “mammal,” as used herein, refers warm-blooded placental animals with hair and with mammary glands capable of producing milk. Mammals include cattle, buffalo, other bovine animals, sheep, goats, pigs, horses, dogs, cats, rats, rabbits, mice, and humans. Also included are other livestock, domesticated animals, captive animals and sports animals. The term “farm animals” includes chickens, geese, emus, fish, ostriches, turkeys, and other farmed animals.

“Micro-algae” refers to single-celled microscopic plant-like organisms such as phytoplankton or diatoms which may form aggregates such as mats or colonies. (They are distinct from polycellular algae such as seaweeds).

“N-3” refers to the group of omega-3 fatty acids having the first double bond located three carbon residues from the omega end of the molecule. N-3 HUFA refers to the group of n-3 highly unsaturated fatty acids with five or more double bonds (e.g. EPA, DPA, and DHA).

“DHA” refers to 4,7,10,13,16,19-docosahexaenoic acid (22:6n-3).

“DHAOIL” refers to a composition of matter comprising substantially only DHA in its n-3 HUFA fraction.

“EPA” refers to 5,8,11,14,17,-eicosapentaenoic acid (20:5n-3).

“EPAOIL” refers to a composition of matter comprising substantially only EPA in its n-3 HUFA fraction.

“CLA” refers to conjugated linoleic acid, a fatty acid with a carbon chain length of 18 and two double bonds in the cis 9 trans 11 conformation.

As used herein, a “solely heterotrophic culture” is a culture produced without light. (e.g. “Heterotrophic diatoms” are those diatoms capable of growing in the dark on a particular carbon substrate). As used herein, a “primarily heterotrophic culture” is determined as follows: i) a culture (culture A) is produced mixotrophically (with both organic carbon sources in the medium as well as a light source, ii) under identical conditions a culture (Culture B) is produced but without the light (solely heterotrophically), iii) dry weights are obtained from both cultures, iv) dry (washed) biomass is analyzed for carbon content, and v) the percentage contribution of light to the process is estimated by a) taking the value for carbon in washed dried biomass of Culture A, b) subtracting the value for carbon in that of Culture B, c) dividing by the value of Culture B, and d) multiplying by 100%. The value obtained is only an estimate but we consider that the estimate will be if anything a high estimate as it is conceivable that the addition of light enhances heterotrophic efficiency. Substantially heterotrophic is then defined herein a process where the percentage obtained through applying the formula (above), is less than about 50%, preferably is less than about 40%, more preferably less than about 20%, and still more preferably less than about 10%.

As referred to herein, “productivity” with respect to a total fatty acid yield can be determined as the biomass dry weight obtained from a given culture volume, or in the case of a continuous culture, from a given culture volume over a set period of time multiplied by the percentage total fatty acid in the biomass.

As used herein “treatment” or “treating” an animal (e.g. human, or other animal) includes administering a composition, formulation, food product, or nutritional supplement in order to ameliorate, prevent, or improve a condition, disease, the physiology, or the health status of a subject.

The description of the invention to be provided herein is given purely by way of example and is not to be taken in any way as limiting the scope or extent of the invention.

Throughout this specification, unless the text requires otherwise, the word “comprise” and variations such as “comprising” or “comprises” will be understood to imply the inclusion of a stated integer or step or group of integers or steps but not the exclusion of any other integer or step or group of integers or steps.

### Compositions

Certain compositions provided herein comprise between about 10% and about 50% by weight n-3 HUFA fatty acids, less than about 15% myristic acid, and less than about 20% pannitic acid. The n-3 HUFA fatty acids in these embodiments may comprise EPA, for example where the ratio of EPA to other n-3 HUFA fatty acids in the composition is at least 6:1.

A preferred embodiment of a composition may comprise between about 18% and about 50% by weight n-3 HUFA fatty acids, less than about 11% myristic acid, and less than about 20% palmitic acid. The n-3 HUFA fatty acids in these embodiments may comprise EPA, for example where the ratio of EPA to other n-3 HUFA fatty acids in the composition is at least 6:1. In other embodiments, the ratio of C14:0 to EPA in the composition is no more than about 0.66:1 and the ratio of C16:0 to EPA in the composition is no more than about 1:1. Such compositions can be the form of a nutritional supplement.

The total fatty acids profile of certain compositions may comprise between about 5% and about 50% EPA, where the ratio of EPA to any other n-3 HUFA present in the composition is at least about 9:1, and where the ratio of C14:0 to EPA in the composition is no more than about 0.66:1 and the ratio of C16:0 to EPA in the composition is no more than about 1:1. In alternative embodiments, the n-3 HUFA fatty acids comprise DHA, and the ratio of DHA to other n-3 HUFA fatty acids in the composition is at least about 9:1, or alternatively where the ratio of C14:0 to DHA in the composition is no more than about 0.33:1 and the ration of C16:0 to DHA in the composition is no more than 0.5:1. Other embodiments of a composition comprise between about 14% and about 50% by weight of DHA, where the ratio of DHA to any other n-3 HUFA present in the composition is at least about 9:1, the ratio of C14:0 to DHA in the composition is no more than about 0.33:1 and the ratio of C16:0 to DHA in the composition is no more than about 0.5:1.

One preferred embodiment of composition comprises at least about 18% by weight EPA; less than about 20% palmitic acid (C16:0); less than about 11% myristic acid (C14:0); and at least about 0.1% alpha linolenic acid (18:3 n-3), wherein the ratio of EPA to DHA is at least about 6:1.

The compositions can be in the form of a nutritional supplement. Such compositions may be formulated to be suitable for non-aquaculture animal feed supplement, for example where composition is manufactured by a process involving heterotrophic or primarily heterotrophic production of micro-algae. Such embodiments include compositions where no further lipid processing or purification steps are made to a biomass comprising the n-3 HUFA fatty acids, as well as embodiments of compositions where additional steps of processing or purification are made to attain the composition profiles described above.

Other preferred embodiments include compositions where no further lipid processing or purification steps are made to the biomass comprising the n-3 HUFA fatty acids, other than steps to extract of an oil comprising the total lipid of the biomass and enhancing the smell taste, texture color and stability of the oil without substantially altering its fatty acid composition.

Some particular embodiments are directed to a composition, including a nutritional supplement, where the ratio of EPA to any other n-3 HUFA present in the composition is at least about 5:1, where the ratio of EPA to any other n-3 HUFA present in the composition is at least about 6:1, where the ratio of EPA to any other n-3 HUFA present in the composition is at least about 7:1, where the ratio of EPA to any other n-3 HUFA present in the composition is at least about 8:1, where the ratio of EPA to any other n-3 HUFA present in the composition is at least about 9:1, where the ratio of EPA to any other n-3 HUFA present in the composition is at least about 10:1, where the ratio of EPA to any other n-3 HUFA present in the composition is at least about 15:1. Certain embodiments are directed to a composition, including a nutritional supplement, where the ratio of EPA to DHA in the composition is at least about 5:1, where the ratio of EPA to DHA in the composition is at least about 6:1, where the ratio of EPA to DHA in the composition is at least about 7:1, where the ratio of EPA to DHA in the composition is at least about 8:1, where the ratio of EPA to DHA in the composition is at least about 9:1, where the ratio of EPA to DHA in the composition is at least about 10:1, where the ratio of EPA to DHA in the composition is at least about 15:1. Such embodiments include compositions where no further lipid processing or purification steps are made to a biomass comprising the n-3 HUFA fatty acids, as well as embodiments of compositions where additional steps of processing or purification are made to attain the composition profiles described above.

Certain embodiments are directed to a composition comprising at least 10% by weight EPA, at least 12% by weight EPA, at least 13% by weight EPA, at least 14% by weight EPA, at least 15% by weight EPA, at least 16% by weight EPA, at least 17% by weight EPA, at least 18% by weight EPA, at least 19% by weight EPA, and at least 20% by weight EPA. Certain embodiments are directed to a composition, including a nutritional supplement, comprising between about 15% and about 20% by weight EPA, comprising between about 10% and about 30% by weight EPA, comprising between about 10% and about 50% by weight EPA, comprising between about 20% and about 50% by weight EPA, between about 30% and about 50% by weight EPA, or between about 40% and about 50% by weight EPA. Such embodiments include compositions where no fuher lipid processing or purification steps are made to a biomass comprising the n-3 HUFA fatty acids, as well as embodiments of compositions where additional steps of processing or purification are made to attain the composition profiles described above.

Certain embodiments are directed to a composition comprising less than 15% myristic acid, less than 14% myristic acid, less than 13% myristic acid, less than 12% myristic acid, less than 11% myristic acid, less than 10% myristic acid, less than 8% myristic acid, less than 15% palmitic acid, less than 12% palmitic acid, or less than 10% palmitic acid.

Certain embodiments are directed to a composition described herein further comprising at least about 0.05% alpha linolenic acid (18:3 n-3); or at least about 0.1% alpha linolenic acid (18:3 n-3); or at least about 0.5% alpha linolenic acid (18:3 n-3).

In some embodiments, the yields for continuous cultures according to certain methods described herein are at least about 0.2 grams of total fatty acids per liter per day, at least about 0.3 grams of total fatty acids per liter per day, at least about 0.4 grams of total fatty acids per liter per day, at least about 0.5 grams of total fatty acids per liter per day, at least about 0.6 grams of total fatty acids per liter per day, at least about 0.7 grams of total fatty acids per liter per day, at least about 0.8 grams of total fatty acids per liter per day, or at least about 1.0 grams of total fatty acids per liter per day. In some embodiments, the yields for continuous cultures according to certain methods described herein are between about 0.3 grams and about 1.0 grams of total fatty acids per liter per day. between about 0.4 grams to about 1.0 grams of total fatty acids per liter per day, and between about 0.5 grams and about 1.0 grams of total fatty acids per liter per day. In other specific embodiments, the yields for continuous cultures according to certain methods described herein are between about 0.39 grams to about 0.78 grams of total fatty acids per liter per day. In other specific embodiments, the yields for continuous cultures according to certain methods described herein are between about 0.52 grams and about 1.04 grams of total fatty acids per liter per day.

In certain embodiments, the yields for batch cultures according to certain methods described herein are at least about 2 grams per liter in batch, at least about 5 grams per liter in batch culture, at least about 10 grams per liter in batch culture, and at least about 15 grams per liter in batch culture. In some embodiments, the yields for batch cultures according to certain methods described herein are between about 2 grams and about 5 grams per liter, between about 5 grams and about 10 grams per liter in batch culture, and still more preferably between about 10 grams and about 20 grams per liter in batch culture. In other specific embodiments, the yields for batch cultures according to certain methods described herein are between about 2.4 grams and about 4.8 grams per liter, and preferably between about 4.8 grams and about 9.4 grams per liter in batch culture, and still more preferably between about 9.4 grams and about 18.6 grams per liter in batch culture.

### Methods of Production

DHAOILs derived from microalgal cultures when provided as dietary supplements intended even to provide even a modest eventual DHA intake to humans or food producing animals can constitute a significant contribution to the total C14:0 and/or C16:0 dietary load. For example, when fed to dairy cows at a volume required to incorporate 0.4-0.8 g/L DHA into cows' milk, dietary supplementation with compositions derived from Schizochytrium sp. led to a significant concomitant rise in C14:0 and C16:0 in milk (Franklin et al., J Nutr, 129: 2048-2052 (1999)). U.S. Pat. No. 6,568,351, as an example, provides methods for raising rabbits by supplementing their feed with these compositions which also leads to an increase in C14:0 and C16:0 in meat. Under similar circumstances supplementation with EPAOILs comprising relatively high levels of C14:0 and C16:0 might also be expected to provide a significant contribution to the total C14:0 and/or C16:0 dietary load. Prior to the present invention, technology enabling the targeted development of EPAOILs or DHAOILs with low levels of C14:0 and C16:0 had not been achieved.

The divergent health effects of DHA and EPA indicate that different levels of intake may be beneficial at different life stages, and during different nutritional and disease states. This together with the significantly greater cost of production (by weight of n-3 HUFA) of EPAOILs as compared with DHAOILs mean that different manufactures and different technologies enabling new end uses may be desirable and necessary in order to render these compositions industrially applicable and suitable for general food and dietary supplement use.

Such technologies may include those able to identify suitable microorganisms and screen them to identify their ability to assimilate and grow efficiently on different nutrient and/or light intensities and qualities. It is known that the lipid composition of microorganisms can change significantly under different culture conditions and sometimes in a predictable manner. However, until now this knowledge had not been applied systematically to the search for and identification of microorganisms capable of being involved in a manufacture to producing the desired compositions of the present invention.

Such technologies may be dependent on those able to effect a beneficial change in the composition of specific tissues of non-aquaculture food producing animals by supplementing the diets of such animals with such compositions. These changes may include but not necessarily be limited to a significant increase in the presence one or more highly unsaturated omega-3 fatty acids, a decrease in deleterious saturated fatty acids such as C14:0 and C16:0, and an increase in other beneficial fatty acids such as CLA.

It is believed that prior to the present invention no foods have been produced by supplementing the diets of dairy cows or other large food-producing mammals with any EPAOIL. Furthermore, it is the inventors' understanding that prior to the present invention no foods have been produced by supplementing the diets of any non-aquaculture food producing animal with compositions produced by manufactures involving heterotrophic or primarily heterotrophic production of microorganisms comprising EPAOIL. To our knowledge, prior to the present invention no foods have been produced by supplementing the diets of any non-aquaculture food producing animal with compositions produced by manufactures involving primarily heterotrophic production of microorganisms comprising either EPAOILs or DHAOILs containing relatively low levels of C14:0 and C16:0.

Such technologies may also include those able to produce an effect in non-aquaculture food producing animals or in non-aquaculture non food, producing animals whereby a disease, disorder or complaint is prevented, ameliorated or cured by supplementing the diet of such an animal with an EPAOIL composition. To our knowledge prior to the present invention no studies had been undertaken providing EPAOIL compositions produced by manufactures involving heterotrophic or primarily heterotrophic production of microorganisms and administered to animals in order to study the beneficial health effects of these on the animals concerned.

Such technologies may also include those able to effect a beneficial change in the composition of specific tissues of humans accomplished by supplementing the diets of humans with compositions comprising EPAOILs containing relatively low levels of C14:0 and C16:0 produced by approaches involving heterotrophic or primarily heterotrophic production of microorganisms, or with foods produced by supplementing the diets of food producing animals with such compositions. To the inventors' knowledge, prior to the present invention no dietary supplementation trials have been conducted in humans with such compositions or with any foods produced by supplementing the diets of food producing animals with any EPAOIL.

Such approaches may produce an effect in humans whereby a disease, disorder, or complaint is prevented, ameliorated, or treat by supplementing the diet of humans with an EPAOIL composition or other composition provided herein.

In short, prior to the present invention, methods by which food can be produced by supplementing the diets of non-aquaculture food producing animals with EPAOILs and DHAOILs comprising relatively low levels of C14:0 and C16:0 had not been achieved to a satisfactory level.

US Patents (including U.S. Pat. Nos. 5,397,591; 5,407,957; 5,492,938; 5,547,699; 5,550,156; 5,711,983) disclose manufactures preferentially involving the dinoflagellate microorganism Crypthecodinium cohnii to provide compositions with a C14:0 to DHA and C16:0 to DHA ratios of as low as 0.25:1. While these ratios are desirable, an improvement would be very beneficial for making dietary supplements. Also, the inability of making large scale preparations having the desired lipid profile in addition in combination with the commercially prohibitive cost of producing Crypthecodinium cohnii compositions prevents their general application for food and dietary supplement purposes.

US Patents (including U.S. Pat. Nos. 5,130,242; 5,340,594; 5,340,742; 5,518,918; 5,656,319; 5,698,244; 5,908,622; 6,177,108; 6,410,282; 6,451,567; 6,509,178; 6,566,123; 6,582,941; 6,607,900; 6,566,123) disclose manufacturing approaches preferentially involving the thraustochytrid microorganisms Schizochytrium sp., Schizochytrium SR21 strain, and Ulkenia sp. These report microalgal biomasses comprising ratios of C16:0 to DHA that are regarded as highly undesirable DHAOIL compositions. For example, Martek Biosciences' product specification sheet for its microalgal DHAOIL product DHASCO-S specifies a DHA to C16:0 ratio of 0.59:1 and Australia New Zealand Food Safety Authority Application No. A522 specifies a DHA to C16:0 ratio of 0.71:1 for the microalgal DHAOIL product Nutrinova DHA.

U.S. Pat. No. 5,244,191 reports a heterotrophic or largely heterotrophic manufacture preferentially involving the micro-algal species Nitzschia alba where only three types of fatty acids are produced, thus apparently simplifying the purification process. Such a fatty acid composition may help to make a micro-algal oil suitable for production of purified EPA, however it contains an undesirably high C14:0 to EPA ratio of more than 5.5:1 and it contains an undesirably high C16:0 to EPA ratio of more than 7.6:1, thus making the oil composition totally unsuitable for the purposes of the invention.

A heterotrophic manufacture of the microorganism Nitzschia laevis is disclosed by Wen & Chen, Biotechnol. Prog., 18, 21-28 (2002), which reports compositions allegedly containing substantially only EPA as their n-3 HUFA and relatively low levels of C14:0 and C16:0. To the best of the inventors' knowledge, the work carried out by Wen and Chen did not identify fatty acids including those with a carbon chain length longer than 20 such as DPA and DHA. Wen & Chen and others have considered the organisms to be a potential source purified EPA and others have considered it as a potential source of aquaculture hatchery feed (Tan & Johns, J. Appl. Phycol., 8:59-64 (1996)); Simental-Trinidad, J A et al., J Shellfish Res 20, 611-617 (2001)). These works were not directed to producing a source of EPA-rich compositions for general food and dietary supplement use and improvements are still needed to decrease the C14:0 levels in cultures, to provide cultures where productivity of total fatty acids is high enough to be useful and where ratios of EPA to DHA and/or to other n-3 HUFAs are acceptably high. Also, these approaches have only been carried out on a small scale that is not practical for producing enough material to be useful for making compositions for nutritional supplements. In certain embodiments provided herein, the organism Nitzschia laevis is used to make compositions, but which are not produced according to the methods described by Wen & Chen above or which have lipid profiles which are distinguishable.

The microbial biomass from which the fatty acid composition of the invention can comprise, or originate , can result from the culture of any type of microorganism able to produce an EPAOIL for example a bacterium, a yeast, a fingus or a microalgae (or a mixture thereof). For example, fatty acid composition of the invention can comprise an eicosapentaenoic acid (EPA)-rich oil preferably obtained from microalgae. The currently regarded scientific consensus genera of microalgae fall within the following families of algae; Bacillariophyceae, Bodonophyceae, Chlorophyceae, Chrysophyceae, Cyanophyceae, Dinophyceae, Euglenophyceae, Phaeophyceae, Rhodophyceae, Trichomonadophyceae, Xanthophyceae, and hybrids based on the preceding.

Also contemplated are wild strains, mutants or recombinantly constructed microorganisms which produce increased amounts of EPA when cultured in accordance with the present invention.

Microalgal cells may be obtained from culture collections, or isolated from environmental samples, including coastal oceanic and terrestrial bodies of water and ice, ice, either free, thermal vents living or attachments to other organisms such as seaweeds, corals funghi, forarninifora within these environments.

Suitable FA compositions under photoautotrophic conditions. Heterotrophic or mixotrophic growth produces unsuitable FA comp. These include, Amphidinium carteri; Chaetoceros calcitrans; Chlorella minutissima UTEX 2341; Cylindrotheca fusiformis; Monodus subterraneus UTEX 151; Nannochloropsis oculata; Navicula saprophila; Nitzschia closterium (=P. tricornutum); Nitzschia laevis; Phaedoactylum tricornutum UTEX 640; Phaedoactylum tricornutum UTEX 642; Porphyridium cruentum; Skeletonema costatum. These are described in the following references: Yongmanitchai, W. and Ward, O. Phytochemistry, 30:9. 2963-2967. (1991); Servel et al., Phytochemistry, 36:3, 691-693 (1994); Nanton & Castell Aquaculture, 163: 251-261(1998); Brown et al., Aquaculture, 151: 315-331 (1997); Liang et al., J. Appl. Phycol., 17: 61-65 (2005); Vazhappilly & Chen J. of Am. Oil C1hem. Soc., 75: 393-397 (1998); Kitano et al., J. Appl. Phycol., 9: 559-563 (1997);Kitano et al., J. Appl. Phycol., 10: 101-105 (1998); Tan & Johns J. Appl. Phycol., 8: 59-64 (1996); Fuentes et al., Journal of Biotechnology 70: 271-288 (1999); Pennarun et al., J. Agric. Food Chem., 51: 2006-2010 (2003); Leonardo & Lucas Aquaculture 182: 301-315 (2000); Berge et al., Phytochemistry 39: 5 1017-1021 (1995); Kotzabasis et al., Journal of Biotechnology, 70: 357-362 (1999); Ilyash et al, Izvestiya Akademii Nauk SeriyaBiologicheskaya (4): 472-477 Jul.-Aug. 19 (1996).

Suitable FA compositions under photoautotrophic growth. As far as we know, not able to grow heterotrophically. These include, Amphidinium sp.; Biddulphia sinensis; Chaetoceros muelleri; Heteromastrix rotunda; Monochrysis lutheri; Nannochloropsis-like; Navicula like, species unknown; Navicula sp.; Nitzschia frustulum; Nitzschia paleacea; Pavlova lutheri; Pavlova pinguis; Pseudopedionella sp; Rhodosorus sp.; Cricosphaera carterae. These are described in the following references: Mansour et al., J. Appl. Phycol., 17, 287-300 (2005); Volkman et al., Phytochemistry, 19: 1809-13 (1980); Lokman Arch. Int. Phys. Biochim. Biophys., 101: 253-256 (1993); Leonardos & Lucas Aquaculture 182, 301-315 (2000); Yongmanitchai, W. and Ward, O. Phytochemistry, 30: 9. 2963-2967 (1991); Brown, M. R., et al., Aquaculture, 151: 315-331 (1997); Rao et al., Marine Ecology, 26: 63-71 (2005); Renaud, S. M. et al., J. Appl. Phyc. 7: 595-602 (1995); Delaunay et al., J. Exp. Mar. Biol. Ecol., 173: 163-179 (1993).

EPA-rich but no record of producing suitable compositions. These include, Chlamnydomonas plethora; Coccolithus huxleyi; Cricosphaera carteri; Cryptomonas maculata; Cyclotella cryptica; Dunaliella primolecta; Dunaliella tertiolecta; Emiliania huxleyi; Isochrysis galbana; Nannochloris oculata; Pavlova salina; Pavlova viridis; Porphyridium purpureum; Prorocentrum minimum; Rhodomonas lens; Schizochytrium aggregatum; Tetraselmis suecica; Thalassiosira pseudonana; Nannochloropsis sp. These are described in the following references: Rao et al., Mar. Ecol. 26: 63-71 (2005); Galloway J. Phycol., 26: 752-760 (1990); Sriharan et al., Appl. Biochem. Biotech., 28: 317-326 (1991); Nanton & Castell Aquaculture, 163: 251-261 (1998); Nanton & Castell Aquaculture 175: 167-181 (1999); Delaunay et al., J. Exp. Mar. Biol. Ecol., 173:163-179 (1993); Riebesell et al., Geochim. Cosmochim. Act., 64: 4179-4192 (2000); Yamamoto et al., Org. Chem., 31: 799-811 (2000); Bell & Pond Phytochemistry. 41: 465-471 (1996); Thompson et al., J. Phycol., 28: 488-497 (1992); Nuutila et al., J. Biotech., 55: 55-63 (1997); Ohta et al., J. Ferm. Bioeng., 74: 398-402 (1992); Ohta et al., Botanica Marina, 36: 103-107 (1993); Jonasdottir Mar. Biol. 121: 67-81 (1994); Delaporte et al., Comp. Biocehm. Physiol., 140: 460-470 (2005); Thompson et al., Aquaculture, 143: 379-391 (1996); Zhukova Russian Journal of Plant Physiology, 51: 702-707 (2004); Wood et al., Journal of Biotechnology 70: 175-183 (1999); Grima et al, Process biochemistry 27: 299-305 (1992); Servel et aL, Phytochemistry, 36:3 691-693 (1994); Otero et al., Biotechnol. Appl. Biochem. 26: 171-177 (1997); Fidalgo et al, Aquaculture. 166: 105-116 (1998); Volkman et al., Phytochemistry, 30: 6, 1855-1869 (1991); Li et al., Annals of Microbiology, 55, 51-55 (2005); Fuentes et al., Journal of Biotechnology. 70: 271-288 (1999); Rogova J. Plant Physiol. 149: 241-245 (1996); Grima Appl. Microbiol. Biotechnol. 38: 599-605 (1993); Adlerstein & Bigogno J. Phycol. 33: 975-979 (1997); Sukenik et al., Aquaculture, 117: 313-326 (1993).

If the set of genes providing for synthesis of EPA were to be taken from an organism possessing same, and transferred into another organism, such as a plant or animnal, along with gene control mechanisms providing for expression of said genes at a reasonable level, then it might no longer be necessary to rely on micro-algae as the original source of EPA.

Using aseptic technique, cells or aggregates of cells may be physically separated from isolates sourced from environmental samples or culture collections by methods well known to those in the art. For instance, live single cells can be isolated by use of microscope and micropipette and subsequently cultured in the appropriate medium, or growing monoclonal colonies can be transferred from agar plates streaked with a sample from nature or culture. Alternatively various antibiotics may be applied (to cultures of eukaryotic algae) in order to terminate unwanted bacteria (Andersen & Kawach, Chapter 6 in Algal Culturing Techniques (ed. Andersen) Elsevier (2005)).

Culturing of microalgae is normally done in liquid media. The most commonly used media are often based on natural or artificial seawater with additional micro- and macronutrients to assist growth. Macronutrients for most microalgae are nitrogen and phosphorous, and in the case of diatoms also silicate. The latter is usually supplied in the form of sodium metasilicate either in its 5 or 9 hydrate form, for instance at a concentration of 120 mg per liter in the culture medium. Nitrogen may be supplied in a form or forms suitable for use by microalgae in the biosynthesis of nitrogen containing molecules (i.e. sodium nitrate, potassium nitrate, yeast extract, tryptone, corn steep liquor, urea, ammonia). An example is the provision of sodium nitrate as a sole nitrogen source at between 30 mg and 1200 mg per liter of culture medium. Phosphorus is normally added as phosphate, which is readily assimilated by algae. Micronutrients or trace metals (i.e. iron, manganese, zinc, and cobalt) are added to the media in low concentrations (<<1 mg per liter). Most media also contain vitamins, usually B12, thiamine and biotin, although most algae require only one or two of them (Provasoli & Carlucci in Algal Physiology and Biochemistry (ed. Stewart) Blackwell Scientific, UK (1974)).

In order to enable substantially heterotrophic growth, one or more sources of organic carbon is also be supplied to the culture. A variety of monosaccharides, disaccharides and polysaccharides may be used to provide organic carbon in an assimilable form in the culture medium. Sources including relatively pure fructose, glucose, lactose, maltose and sucrose, or combinations of these may be employed depending on the microorganism used. An example is the provision of glucose at concentrations of 2, 5, , 25 or 50 grams per liter in the culture medium (Wen & Chen Journal of Industrial Microbiology and Biotechnology, 25: 218-224 (2000)). Complex carbon sources may also be utilized, including but not limited to: raw cane or beet sugar, starches or industrial byproducts containing sugars such as corn steep liquors which also contain complex nitrogen sources. Whey streams containing lactose have been found to be particularly useful for the certain methods of the invention.

Nutrients may be supplied in the medium at the beginning of a production cycle or strategies well known to those of skill in the art may also be used to regulate the dilution of growth medium and the replacement of nutrients in the culture. Culture media recipes are numerous and easily accessible either from culture collection homepages (i.e. UTEX Culture Collection of Algae http://www.bio.utexas.edu/research/utex/) or published literature (i.e. Starr & Zeikus J. Phycol. 29: 1-107 (1993); Andersen et al., (2005) Appendix A in Algal Culturing Techniques (ed. Andersen) Elsevier).

In order to avoid contamination during cultivation, the culture medium and all equipment (culture vessels, pipettes, inoculum loops etc.) used when working with the growing cultures should be sterilized prior to inoculation. This can be achieved by various techniques, all known to those skilled in the art. Typically used methods for sterilizing equipment are autoclaving (steam pressure at 2 atm. and 121 DEG C) or flaming (passing items through an open flame). Medium and stock solutions of nutrients are sterilized either by autoclaving or filtration (<0.2 μm pore size filter) (Kawachi & Noel Chapter 5 in Algal Culturing Techniques (ed. Andersen) Elsevier (2005)).

The cultivation can be carried out at any temperature at which the microalgal species can be grown. Typically the media in the reactor is kept at a temperature of 25 to 40 degrees via the use of a heat exchange mechanism. Such mechanisms are well known to those skilled in the art. EPA is generally increased as a proportion of total fatty acids at lower temperatures but higher temperatures may increase overall productivity of EPAOIL. A convenient, and economical, temperature to carry out the cultivation for N. laevis is 20 DEG C.

The pH value of the culture medium should be controlled. Typically a pH of 7 to 9 is preferred by microalgal strains, but higher or lower pH levels may used, depending on the microalgae and the composition of the medium, as high and low pH values may cause precipitation of nutrients. The pH may be controlled throughout the process of cultivation by the addition of a suitable acid or base, such as hydrochloric acid, sodium hydroxide, potassium hydroxide, and the like. For the cultivation of Nitzschia laevis it is preferred to maintain the pH at between 7.5 to 8.5 (Wen & Chen, Biotechnol. Bioeng., 75:159-169 (2001)).

For long-term preservation of cultures, stock cultures are kept either on agar slants or in liquid medium under conditions sub-optimal for growth (i.e. low temperature) (Lorenz et al., Chapter 10 in Algal Culturing Techniques (ed. Andersen) Elsevier (2005)). Alternatively, a variety of cryogenic preservation techniques can be employed to provide for storage of viable cells over long periods of time. (See J. G. Day et al., J Appl Phycology 9: 121-127, (1997), and J. G. Day et al., J Appl Phycology 12: 369-377, (2000)). Inocula may be prepared by transferring cells from agar slants or plates to tubes containing 6-10 ml of culture medium. After a period of growth, the cells in the tubes are in turn used to inoculate 200 ml of sterile growth medium in a 500 ml shake flask. The contents of the inoculated shake flask are used to inoculate a small mechanically agitated fermentor of up to several liters in volume. Larger production reactors are then inoculated at the beginning of a production cycle with inocula prepared by the method above at about 10% of the volume of the reactor.

In fed batch, continuously fed batch, or continuous processes nutrients may be replenished by addition of fresh medium or topped up by means of addition of concentrated stock solutions. It is known to those skilled in the art that alteration of nutrient concentrations and ratios in media may affect lipid production and composition in microalgae. In particular, significant work has been done on altering the nitrogen to carbon ratio and the availability of nitrogen and silicate in microalgal cultures at different parts of the culture cycle. For example limiting the availability of silicate to diatoms is known to trigger oleogenesis (Borowitzka, “Micro-Algal Biotechnology”, Cambridge University Press (1988)). U.S. Pat. No. 5,244,191 discloses a method of inducing an oleogenic phase in Nitzschia alba via a specifically timed imposition of silicate deficiency and that the length of time of the oleogenic phase will depend on the type of microorganism cultivated and the available nutrient supply. U.S. Pat. No. 5,244,191 however does not suggest how the timing of any nutrient deficiency should be adjusted for any particular microorganism, nor do they teach how either the timing, length or specific combination of any nutrient deficiency may alter the relative composition of lipids in any microorganism. In the present invention the culture process may be controlled by altering the ratios of and availability of key nutrients either at the beginning of, in increments, or continuously throughout the culture cycle or as part of a fmishing step to ensure that the resulting fatty acid composition remains within the specifications provided for an EPAOIL or a DHAOIL. For example it may be beneficial to utilize different carbon substrates with different species in order to produce an EPAOIL. It is known that for certain organisms, fatty acid compositions may be beneficially altered by utilization of different carbon substrates in the culture medium see for example Wu, Yu & Lin, “Effect on culture conditions on docosahexaenoic acid production by Schizochytrium sp. S31. Process Biochemistry, 40: 3103-3108 (2005). By undertaking a systematic approach to determining the effect of carbon sources on microorganisms it may be possible, when other parameters are also taken into account simultaneously, to determine which combinations may be able to lead to the production of EPAOILS or DHAOILS with relatively low levels of C16:0 and C14:0. A preferred carbon source for the production of EPAOILS with relatively low C16:0 and C14;0 using the microorganism Nitzschia laevis is lactose.

Light is another factor of great importance to the growth and cellular composition of microalgae, and a strategy for enhancing fatty acid production efficiency and/or evoke a physiological response commensurate with improving the fatty acid composition of the biomass, is to expose the micro-algal cells to light varying in intensity, frequency and duration. Various authors have studied the effects of light intensity and wavelength on lipid production in photosynthetic cultures of rnicroalgae and related organisms (see: Mock & Kroon, Phytochemistry 61: 5-60 (2002); Saavedra & Voltolina Comparative Biochemistry and Physiology B—Biochemistry & Molecular Biology, 107: 3944 (1994); Radwan et al., Z. Naturforsch, 43c: 15-18 (1988)). A number of papers describe the mixotrophic production of microalgae where light is the predominant energy source (i.e. Kotzabasis et al., Journal of Biotechnology, 70: 357-362 (1999)). Very few, if any, studies however have reported on the effect of exposing lipid-producing microorganisms in primarily heterotrophic cultures to relatively small amounts of light. Neither the above-mentioned authors nor any prior art publications have shown how very low levels of light or restricted wavelengths of light or both in combination can be used to produce EPAOILS or DHAOILS in substantially heterotrophic commercial cultures of microalgae. It may be useful to construct bioreactors partially or in whole from transparent materials, so that cells can be efficiently exposed to these low amounts of light. A large number of photobioreactor designs are known to those of skill in the art, as reviewed in Su & Lee Biotechnology and Bioprocess Engineering, 8: 313-321 (2003).

Preferably, the total amount of light each cell is exposed to will be low enough to minimize the electricity consumption in the case of artificial light and in the case of natural sunlight the proportion of the culture at any given time that may need to be in contact with transparent reactor surfaces so as to minimize the need for these in bioreactor construction. The need for cells to be exposed to only very low light levels will ideally reduce any unwanted variation that natural light intensity and duration may have on the lipid productivity and composition of a cultured biomass by restricting exposure of cells to the lowest common duration and intensity of light available or less. In order to achieve low total cell exposure, cells may be exposed to low intensity light continuously or higher intensity light periodically or a combination. An effect known as light:dark cycling may be achieved either by mixing cells within a reactor where a number of light zones and dark zones may exist, via exposure of parts of the reactor to light sources of varying intensity and/or duration, or a combination of these methods. Alternatively, cells can be exposed to restricted wavelengths of light such that the total lipid productivity increases, and/or the proportion of at least one n-3 HUFA in total fatty acids increases and/or the proportion of at least one saturated fatty acid in total fatty acids decreases. In an alternative production method, microorganisms in largely heterotrophic cultures may be exposed over time to a very low amount of light to produce cultures of biomass with high levels of at least one particular n-3 HUFA and relatively low levels of C16:0 and/or C14:0. By undertaking a systematic approach to determining the effect of low levels of light intensity and/or restricted wavelengths of light on microorganisms it may be possible, when other parameters are also able to be taken into account simultaneously, to determine which combinations may be able to lead to the production of EPAOILS or DHAOILS with relatively low levels of C16:0 and C14:0. A preferred method for the production of EPAOILS with relatively low C16:0 and C14;0 using the microorganism Nitzschia laevis is the use of low intensity light providing an equivalent photon flux density to cells to that achieved by providing continuous illumination with white light of 1 to 10 micromol photons per square meter per second to the lower vertical face of a well agitated 500 ml Erlenheyer flask with 200 ml solution of cells of a biomass density of less than one gram of dry biomass per liter. Methods for estimating the per cell light exposure in transparent bioreactors are known to those of skill in the art, i.e. Zhang & Richmond, Biotechnol., 5: 302-310 (2003).

One or several reactors, typically with a capacity of 20-200,000 litres or more, are suitable for the preparation of cultures. While any type of fermentor can be used with the present invention, stainless steel fermentors with 4, 5 or 6-bladed Rushton-type impellors coupled to an electric motor via a rotating a shaft are preferred in some embodiments to provide a high level mixing and maintain a high concentration of dissolved gasses. Parameters for use and design of such reactors are well known by those skilled in the art (M. J. Kennedy “A review of the design of reaction vessels for the submerged culture of micro-organisms”. Industrial Processing Division, New Zealand Dept. of Scientific and Industrial Research (1984)). A preferred fermentor for large-scale cultivation is an air-lift fermentor. In these fermentors, the cultures are agitated via the injection of air, in combination with a geometric design, optimized to regulate hydrodynamic turbulence and gas mass transfer to produce sufficient volumes of microalgal biomass (see: Chisti M. Y., Airlift Bioreactors Elsevier Applied Science (1989)). Air-lift fermentors may reduce energy requirements for mixing. Hybrid partially impellor driven air-lift reactors are also preferred in some embodiments. Concentration of gasses within a reactor may be controlled via a variety of methods known to those of skill in the art including but not limited to injection of filtered air or purified gasses, sparging, control of impellor speed, alteration of reactor design geometry, pressurization of reactors and through the use of a degassing apparatus. Where impellors or air bubbles are used to assist dispersion of gasses and mixing of media within a vessel high impellor tip speeds in media and bursting bubbles at the surface may expose microorganisms to shear stress. These factors should be optimized through design and operating conditions and are able to be optimized by those skilled in the art, if necessary with references to publications referenced within this document. Strategies well known to those of skill in the art may be employed with both types of reactor to reduce the potential for shear damage to shear-sensitive microorganisms. Such strategies include but are not limited to keeping the tip speed of impellers low and employing chemical agents to reduce the viscosity of the culture medium.

Reactors may be run on the lines of a number of variations on the basic themes of batch or continuous cultivation. An example of a continuous culture is described in Z. Y. Wen and S. F. Chen in Biotechnol. Prog. 18: 21-28 (2002). The adoption of sophisticated batch culture strategies such as continuous feeding, dilution and cell recycling, may also have the advantage of achieving finer control of nutrient levels throughout the process as well as removing substances that inhibit growth from the medium resulting in continued growth at high cell densities. (See also: Wen, Z. and Chen, F, Process Biochemistry 38: 523 529 (2002). Alternatively the medium may also be treated by mechanical, chemical or other physical means so as to remove antialgal or autoinhibitory metabolites or to alter the production of secondary metabolites that inhibit growth or productivity of the cells.

Certain embodiments also relate to a method of treating the microbial biomass; here the microbial biomass can be pretreated by a variety of methods well known to those in the art before extraction of the oil. Such methods include flocculation, settling, centrifugation, pasteurization, extrusion, freeze drying, belt drying, spray drying. Cells harvested during or at the termination of the culture may tend to form aggregates and settle out easily or the process of sedimentation may be assisted by flocculation. The following references may be useful: Mackay, D. (1996). Broth conditioning and clarification, Downstream processing of natural products. A practical handbook. (Ed. M. S. Verral), 11-40, John Wiley & Sons; Boonaert, C. J. P et al., “Cell separation, Flocculation: Encyclopedia of Bioprocess Technology” (1999). Fermentation, Biocatalysts and Bioseparation (eds M. C. Flickinger & S. W. Drew), 1: 531-47, John Wiley & Sons, Inc.; Hee-Mock, O. et al., “Harvesting of Chlorella vulgaris using a bioflocculant from Paenibacillus” sp. AM49, Biotechnol. Lett., 23: 1229-34 (2001), each of which is incorporated by reference herein in its entirety.

Alternatively the technique given by M. E. Cartens et al., in J Am Oil Chem Soc 73: 1025-1031 may be followed. Water may be removed from the harvested cells by filtration and/or centrifugation then further dried by drum, spray, or freeze-drying. The methods and techniques described and known in the art relating to centrifugation of cultures include, for example Chisti. Y. & Moo-Young, M. “Fermentation technology, bioprocessing, scale-up and manufacture” (1991). Biotechnology: the science and business (eds M. Moses & R. E. Cape), 167-209. Harwood Academic Publishers, New York; Mackay, D. “Broth conditioning and clarification, Downstream processing of natural products”, A practical handbook, (ed. M. S. Verral), 11-40, John Wiley & Sons (1996); Benemann, J. R. et al., “Development of microalgae harvesting and high rate pond technologies in California” (1980). Algal Biomass (eds G. Shelef & C. J. Soeder), 457. Elsevier, Amsterdam; Richmond, A. & Becker, E. W. “Technological aspects of mass cultivation, a general outline”. In: CRC Handbook of microalgal mass culture (ed. Richmond), 245-63 (1986). CRC Press Inc., Boca Raton, Fla.; D'Souza, F. M. L. et al., “Evaluation of centrifuged microalgae as diets for Penaus monodon Fabricius larvae”, Aquaculture Res., 31: 661-70 (2000); Heasman, M. et al., “Development of extended shelf-life microalgae concentrate diets harvested by centrifugation for bivalve molluscs—a summary”. Special issue: “Live feeds and microparticulate diets”, Aquaculture Res., 31: 8-9, 637-59 (2000); Mohn, F. H. “Improved technologies for the harvesting and processing of microalgae and their impact on production costs”, Archiv fur Hydrobiologie, Beihefte Ergebnisse der Limnologie, 1: 228-53 (1978); Letki, A. G. “Know when to turn to centrifugal separation”, Chem. Eng Prog., September, 29-44 (1998), each of which is incorporated by reference herein in its entirety.

The methods and techniques described and known in the art relating to filtration of cultures include, for example Mohn, F. H. “Experiences and strategies in the recovery of biomass from mass cultures of microalgae”, Algae Biomass (eds G. Shelef & C. J. Soeder), 547-71 (1980); Gudin, C. & Therpenier, C. “Bioconversion of solar energy into organic chemicals by microalgae”, Adv. Biotechnol. Proc., 6: 73-110 (1986); Belter, P. A. et al., “Bioseparations, Downstream processing for biotechnology”, John Wiley & Sons (1988); Ben-Amotz, A. & Avron, M. “The biotechnology of mass culturing of Dunaliella for products of commercial interest. Algal and Cyanobacterial Technology”, (eds R. C. Cresswell, T. A. V., Rees & N. Shah), 90-114, Longman Scientific and Technical Press (1987); and Ruane, M. “Extraction of caroteniferous materials from algae”, Australian Pat. No. 7239574 (1977), each of which is incorporated by reference herein in its entirety.

The methods and techniques described and known in the art relating to spray-, drum- and freeze-drying of cultures and a biomass include, for example Ben-Amotz, A. & Avron, M. “The biotechnology of mass culturing of Dunaliella for products of commercial interest”, Algal and Cyanobacterial Technology (eds R. C. Cresswell, T. A. V., Rees & N. Shah), 90-114, Longman Scientific and Technical Press (1987); Belter, P. A. et al., “Bioseparations. Downstream processing for biotechnology”, John Wiley & Sons (1988); Chisti, Y. & Moo-Young, M. “Fermentation technology, bioprocessing, scale-up and manufacture”, Biotechnology, the science and the business (edsw M. Moses & R. E. Cape), 167-209. Harwood Academic Publishers, New York (1991); Molina-Grima, E. et al., “Gram-scale purification of eicosapentaenoic acid *EPA 20:5n3) from wet Phaeodactylum tricornutum UTEX 640 biomass”, J. Appl. Phycol, 8: 359-67 (1996); Snowman, J. W. “Lyophilization, Downstream processing of natural products”, A practical handbook (ed. M. S. Verral), 275-300, John Wiley & Sons (1996); Bruttini, R. “Freeze-drying, pharmaceuticals”, Encyclopedia of Bioprocess Technology (eds M. C. Fickinger & S. W. Drew) 1276-98,vol.2. John Wiley & Sons (1999), each of which is incorporated by reference herein in its entirety.

Dry or semi dry biomass may be stored in refrigerated vats as a slurry extruded or formed into cakes, pellets or powders. These may then be formulated into food products for human or animal consumption. Certain embodiments also relate to a methods of isolating a polyunsaturated fatty acid-containing oil from a microbial biomass. EPAOIL can be extracted from biomass wet or dry, according to techniques known to those of skill in the art, to produce a complex containing lipids. Total lipids can be extracted from dried or wet biomass by extraction using volatile organic solvents such as ethanol or hexane or supercritical gas chromatography.

The methods and techniques described and known in the art relating to the concentration of extracted lipids to produce intermediate or higher purity EPA oil fractions include, for example those described in Robles Medina, A. et al., “Concentration and purification of stearidonic, eicosapentaenoic and docosahexaenoic acids from cod liver oil and the marine microalga Isochrysis galbana”, JAOCS, 72: 575-83 (1995); Cartens, M. et al., “Eicosapentaenoic acid (20:5n3) from the marine microalga Phaeodactylum tricornutum”, J. Am. Oil Chem. Soc., 73: 1025-31 (1996); Molina-Grima, E. et al., “Gram-scale purification of eicosapentaenoic acid EPA 20:5n3) from wet Phaeodactylum tricornutum UTEX 640 biomass”, J. Appl. Phycol., 8: 359-67 (1996); Ibanez Gonzalez et al., “Optimization of fatty acid extraction from Phaeodactylum tricornutum UTEX 640 biomass”, J. Am. Oil Chem. Soc., 75: 1735-40 (1998); Gimenez, A. et al., “Downstream processing and purification of eicosapentaenoic (20:5n3) and arachidonic (20:4n6) acids from the microalga Porphyridium cruentum. Bioseparation”, 7: 89-99 (1998); Belarbi, H. et al., “A process for high and scaleable recovery of high purity eicosapentaenoic acid esters from microalgae and fish oil”, Enzyme Microb. Technol., 26: 516-29 (2000); Rodiguez-Ruiz, L. et al., “Rapid simultaneous lipids extraction and transesterification for fatty acid analysis”, Biotechnol. Tech. (1998); Belarbi, E. H. et al., “Downstream processing of algal oils rich in polyunsaturated fatty acids: The purification of eicosapentaenoic acid (EPA) with a view towards industrial production”, Algal Biotechnology, A Sea of Opportunities (2002). The 1st Congress of the International Society for Applied Phycology, 9th Imternational Conference on Applied Algology, Aguadulce, Roquetas de Mar, Almeria, Spain, 26-30 May (eds M. Garcia, E. Molina, F. C. Acien, J. M. Fernandez, J. A. Sanchez & C. Brindley, p 106, Universidad de Almeira; Walker, “Supercritical CO2 extraction of Lipids from Pythium irregulars”. J. Am Oil Chem Soc 76: 595-602 (1999). Optimization of supercritical CO2 may be made in light of Yener “Estimation of lipid properties related to supercritical fluid extraction” Int J Food Properties 4:1 45-57 (2001) which leads towards separation of types of lipid, each of which is incorporated by reference herein in its entirety.

Bulk extracted lipids may be processed further immediately or stored prior to further processing. To prevent oxidation of fatty acids during storage antioxidants may be added, oil may be cooled, protected from exposure to oxygen by sparging with an inert gas such as nitrogen or argon, protected from light by dark storage. For lipids extracted using hexane the following reference may be of use to practitioners: Guil-Guerrerro J L “Hexane reduces peroxidation of fatty acids during storage” Eur J Lipid Sci Technol 103: 271-278 (2001). Oils may be further processed into powders, gels, microencapsulated or soft gel encapsulated, blended with existing foods, food ingredients or nutraceuticals, further processed to alter color, flavor and other sensory characteristics.

In certain embodiments, the resulting material from fermentation (which is often called the broth) can be used. Suitably a large proportion of the water is removed, in order to obtain a biomass cake. The biomass at this stage preferably has a dry matter content of from 20 to 75%. The biomass can then be granulated into granular particles. This is preferably achieved by extrusion, which may be preceded by a high shear mixing. Depending on what further processing is anticipated it may be preferable that cell disruption is either prevented or minimized especially if the resulting granules are to be stored prior to further use.

Extracted EPAOIL can be used in that state without further processing, or it can be subjected to one or more further refining steps. Refining of the oil can be performed using standard techniques. For example, the oil can be subjected to degumming, deacidification, bleaching and/or deodorizing. The oil may be relatively stable or alternatively an antioxidant mixture may be added to prevent and or mitigate the effect of oxidative degradation.

### Administration

In another aspect, the compositions described herein can be given to animals, including for example humans or animals which are typically used to provide food products for human consumption.

In certain embodiments the consumption of the compositions by the animals, for example either by direct human consumption or administration, direct animal consumption or administration, or through the consumption of a food product, results in an improvement or amelioration of undesired biochemical, physiological, or physical disease or condition in a subject.

Generally, the compositions described herein are useful for treating a wide range of conditions and disorders, examples of which include impaired glucose tolerance; impaired fasting glucose; diabetes; including type 1 and type 2 diabetes and their complications; insulin resistance; diabetes-associated hypertension; Syndrome X; cancer; osteoarthritis; autoimmune diseases; rheumatoid arthritis; inflammatory and auto-immune diseases other than arthritis; respiratory diseases; neurological disorders; neurodegenerative disorders; renal and urinary tract disorders; cardiovascular disorders; cerebrovascular disorders; degenerative diseases of the eye; psychiatric disorders; reproductive disorders; visceral disorders; muscular disorders; metabolic disorders; prostatic hypertrophy and prostatitis; impotence and male infertility; mastalgia; male pattern baldness; osteoporosis; dermatological disorders; dyslexia and other learning disabilities; and cancer cachexia; obesity; ulcerative colitis; Crohn's disease; anorexia nervosa; bums; osteoarthritis; osteoporosis; attention deficit/hyperactivity disorder; and early stages of colorectal cancer; lung and kidney diseases; and disorders associated with abnormal growth and development, comprising administering to a patient in need thereof an effective amount of the composition.

Other targeted diseases, disorders, and conditions, include cardiomyopathy, including diabetic cardiomyopathy; atherosclerosis; coronary heart disease; hyperglycemia, hypercholesterolemia (e.g., elevated cholesterol in low-density lipoprotein (LDL-C)), hypertension, hyperinsulinemia, and/or hyperlipidemia; diseases and disorders characterized in part by any one or more of hyperlipidemia, hypercholesterolemia (e.g., elevated cholesterol in low-density lipoprotein (LDL-C)), hyperglycemia, hypertension, and/or hyperinsulinemia; diseases, disorders or conditions characterized in whole or in part by elevated LDL-C; and, diseases, disorders or conditions characterized in whole or in part by (a) hypercupremia and/or copper-related tissue damage and (b) hyperglycemia, insulin resistance, impaired glucose tolerance, and/or impaired fasting glucose, and/or elevated or undesired levels of LDL-C, or predisposition to, or risk for, (a) and (b).

While not intending to being limited to any mechanism, methods of treating an animal with the compositions provided herein are believed, in certain cases, and at least in part, to be achieved through a modulation of the lipid composition or metabolism in the animal (e.g. human, cow, etc.). One such improvement is directed to the lipid composition of the serum of a human subject which is correlated with the improvement or amelioration of associated medical conditions. Thus, in certain embodiments a method of altering the serum lipid profile of an animal is provided.

Changes in fatty acids in relevant tissues of humans or animals may be measured via a variety of techniques well known to those of skill in the art. They may include but are not limited to the methods described herein below. Free fatty acids may be analyzed and quantified in plasma by gas-liquid chromatography (hereinafter, “GLC”), following extraction, for example as described in Dol, (1956), J. Clin. Invest. 35: 150; Turnell et al., (1980), Clin. Chem. 26: 1879. Serum triglycerides may be measured following hydrolysis by a mixture of lipase and esterase, with determination of glycerol by kinetic fixed-time analysis additionally using glycerol kinase, pyruvate kinase, and lactate dehydrogenase (see, e.g., Ziegenhom, (1975), Clin. Chem. 2: 1627; Klotzsch & McNamara, (1990), Clin. Chem. 36:1605). The fat composition and/or cholesterol composition of the milk from animal, for example a cow, can be used to determine whether an individual cow produces milk with a suitable composition. Means for obtaining a representative milk sample are well known in the art. The milk sample may be frozen, or may be subjected to further analysis without freezing. The fat composition of the milk sample is measured using methods well known in the art as described infra, and the type and quantity of fatty acids and/or cholesterol present in the milk sample can be recorded. Most often, the individual cow is fed a conventional diet, e.g., for at least about three days, and preferably at least about five days prior to the collection of the milk sample. Methods for determining the type and quantity of fats and fatty acids are described, for example in Cook et al., J. Dairy Res. 39: 211 (1972); Noakes et al., Am. J Clin. Nutr. 63:42 (1996); U.S. Pat. No. 6,242,013. Typically, total fat is determined by extraction from a tissue or fluid, such as milk (or butter made from the milk), by mixing or homogenizing with a suitable solvent such as chloroform, chloroform/ethanol or chloroform/isopropanol, diethyl ether, or petroleum ether, or mixtures such as NH.sub.4OH/ethanol/diethyl ether/petroleum ether (Walstra & Mulder, Neth. Milk Dairy J 18: 237 (1964)), followed by gravimetric analysis. Alternate volumetric methods employ H.sub.2SO.sub.4 to liberate fat, which is then measured. See, e.g., Ling, A Textbook of Dairy Chemistry, 3.sup.rd ed. Vol. 2, Practical, Chapman Hall, London (1956); Horwitz, ed., Official Methods of Analysis, 13 .sup.th ed., Association of Official Analytical Chemists, Washington, D.C. (1980). Rapid determination of the amount of fat in milk can be done by measurement of the absorption of infrared radiation at 3.4 or 5.7 .mu.m (e.g., Horwitz, supra; Goulden,. J. Dairy Res.; 31: 273 (1964)). The fatty acid type and quantity of fat and fatty acids in the extracted fats may be further characterized by chemical cleavage and characterization of fatty acids using, for example, GLC (e.g., James & Martin, Biochem. J. 63:144 (1956); Jensen et al., J. Dairy Sci. 45:329 (1962); Jensen et al., J. Dairy Sci. 50:19 (1967)), in which fatty acids are determined by separation of mixtures of volatile fatty acid derivatives, for example methyl derivatives formed by transesterification with sodium methoxide (Christopherson & Glass, J. Dairy Sci. 52:1289 (1968)). Alternatively, fatty acids may be esterified using sodium butoxide or H.sub.2SO.sub.4 and boron trifluoride catalyzed butyrolysis (Iverson & Sheppard, J Assn Off Anal Chem 60:284 (1977)), enabling determination as butyl esters (e.g., Christopher & Glass, supra; Parodi, Aust. J. Dairy Technol. 25:200 (1970)). Alternatively, milk fatty acids may be determined by GLC-mass spectrometry following argentation thin layer chromatography (hereinafter, “TLC”) (e.g., Strocchi & Holman, Riv. Ital. Sostanze Grasse 48:617 (1971)), or by high resolution open-tubular GLC (e.g., Ackman et al., Lipids 7:683 (1972)). The total amounts of conjugated fatty acids present in milk fat extracts have been determined by ultraviolet spectrophotometry (see, e.g., Smith et al., J. Am. Oil Chem. Soc. 55:257 (1978)). Milk lipid classes from extracts can also be separated and classified by TLC (see, e.g., Smith et al, supra). The cholesterol composition of the milk may be quantified using GLC-mass spectrometry of trimethylsilyl esters (e.g., Mincione et al., Milchwissensch 132:107 (1977)), or by GLC (see, e.g., Parodi, Aust J Dairy Sci 28:135 (1973)). See also, e.g., LaCroix et al., J. Am Diet Assn 62:275 (1973).

### Formulations, Dosages, Treatments, and Methods of Administration

To achieve the desired concentrations in an animal, or in some embodiments a desired effect(s), the n-3 HUFA fatty acids and other bioactive compounds provided herein, may be administered as single or divided dosages, for example, of at least about 1 mg/kg to about 1 g/kg, of at least about 10 mg/kg to about 500 mg/kg, at least about 10 mg/kg to about 300 mg/kg or at least about 5 mg/kg to about 200 mg/kg of body weight or at least about 10 mg/kg to about 200 mg/kg of body weight, although other dosages may provide beneficial results. The amount administered will vary depending on various factors including, but not limited to, the cyclic composition chosen and its clinical effects, the disease, the weight, the physical condition, the health, the age of the mammal, whether prevention or treatment is to be achieved, and if the composition is chemically modified. Such factors can be readily determined by the clinician examining the empirical data from the clinical trials and examining the preclinical animal model results or other test systems that are available in the art.

Administration of the therapeutic agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the compositions of the invention may be essentially continuous over a preselected period of time or may be in a series of spaced doses. Both local and systemic administration is contemplated.

Compositions are prepared via synthesis or otherwise obtained, purified as necessary or desired, and then lyophilized and stabilized. The composition can then be adjusted to the appropriate concentration and optionally combined with other agents. The absolute weight of a given composition included in a unit dose can vary widely.

The n-3 HUFA fatty acids described herein, including EPA and DHA, can be administered separately or in combination with another compound that has a desired bioactivity. They may be packaged separately or be present in the same overall package. Alternatively, using techniques well known to those skilled in the art, n-3 HUFA fatty acids, including EPA and DHA are preferably prepared for administration in a dose in the range of about 0.1 to about 100 g/day, preferably about 1 to about 50 g/day, and still more preferably 0.1 g to 5-10 g/day, in an adult patient of about 70 kg body weight. Dosage forms, for example, tablets and capsules may be prepared accordingly, or using other doses as disclosed herein, and lower doses than those presently prescribed for this agent.

Alternatively, a whole cell biomass can be administered or provided for consumption. Such whole cell biomass, when provided as a food source, are typically provided in a quantity of between about 0.01 kilograms to 10 kilograms a day, 0.1 kilograms to 10 kilograms a day, 0.5 kilograms to 5 kilograms a day, 0.5 kilograms to 3 kilograms a day, or other desired amounts.

Thus, one or more suitable unit dosage forms comprising the n-3 HUFA fatty acids and compositions described herein can be administered by a variety of routes including oral, parenteral (including subcutaneous, intravenous, intramuscular and intraperitoneal), rectal, dermal, transdermal, intrathoracic, intrapulmonary and intranasal (respiratory) routes. The therapeutic compositions may also be formulated in a lipid formulation or for sustained release (for example, using microencapsulation, see WO 94/07529, and U.S. Pat. No. 4,962,091). The formulations may, where appropriate, be conveniently presented in discrete unit dosage forms and may be prepared by any of the methods well known to the pharmaceutical arts. Such methods may include the step of mixing the therapeutic agent with liquid carriers, solid matrices, semi-solid carriers, finely divided solid carriers or combinations thereof, and then, if necessary, introducing or shaping the product into the desired delivery system.

When the therapeutic compositions of the invention are prepared for oral administration, they are generally combined with a pharmaceutically acceptable carrier, diluent or excipient to form a pharmaceutical formulation, or unit dosage form. For oral administration, the compositions may be present as a oil extract, capsule, pill, powder, a granular formulation, a solution, a suspension, an emulsion or in a natural or synthetic polymer or resin for ingestion of the active ingredients from a chewing gum. The active compositions may also be presented as a bolus, electuary or paste. Orally administered therapeutic compositions of the invention can also be formulated for sustained release, e.g., the compositions can be coated, micro-encapsulated, or otherwise placed within a sustained delivery device. The total active ingredients in such formulations comprise from 0.1 to 99.9% by weight of the formulation.

By “pharmaceutically acceptable” it is meant a carrier, diluent, excipient, and/or salt that is compatible with the other ingredients of the formulation, and not deleterious to the recipient thereof.

Pharmaceutical formulations containing the therapeutic compositions of the invention can be prepared by procedures known in the art using well-known and readily available ingredients. For example, the composition can be formulated with common excipients, diluents, or carriers, and formed into tablets, capsules, solutions, suspensions, powders, aerosols and the like. Examples of excipients, diluents, and carriers that are suitable for such formulations include buffers, as well as fillers and extenders such as starch, cellulose, sugars, mannitol, and silicic derivatives. Binding agents can also be included such as carboxymethyl cellulose, hydroxymethylcellulose, hydroxypropyl methylcellulose and other cellulose derivatives, alginates, gelatin, and polyvinyl-pyrrolidone. Moisturizing agents can be included such as glycerol, disintegrating agents such as calcium carbonate and sodium bicarbonate. Agents for retarding dissolution can also be included such as paraffin. Resorption accelerators such as quatemary ammonium compounds can also be included. Surface active agents such as cetyl alcohol and glycerol monostearate can be included. Adsorptive carriers such as kaolin and bentonite can be added. Lubricants such as talc, calcium and magnesium stearate, and solid polyethyl glycols can also be included. Preservatives may also be added. The compositions of the invention can also contain thickening agents such as cellulose and/or cellulose derivatives. They may also contain gums such as xanthan, guar or carbo gum or gum arabic, or alternatively polyethylene glycols, bentones and montmorillonites, and the like.

For example, tablets or caplets containing the cyclic compositions of the invention can include buffering agents such as calcium carbonate, magnesium oxide and magnesium carbonate. Caplets and tablets can also include inactive ingredients such as cellulose, pregelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, zinc stearate, and the like. Hard or soft gelatin capsules containing at least one cyclic composition of the invention can contain inactive ingredients such as gelatin, microcrystalline cellulose, sodium lauryl sulfate, starch, talc, and titanium dioxide, and the like, as well as liquid vehicles such as polyethylene glycols (PEGs) and vegetable oil. Moreover, enteric-coated caplets or tablets containing one or more compositions of the invention are designed to resist disintegration in the stomach and dissolve in the more neutral to alkaline environment of the duodenum.

The therapeutic compositions of the invention can also be formulated as elixirs or solutions for convenient oral administration or as solutions appropriate for parenteral administration, for instance by intramuscular, subcutaneous, intraperitoneal or intravenous routes. The pharmaceutical formulations of the therapeutic compositions of the invention can also take the form of an aqueous or anhydrous solution or dispersion, or alternatively the form of an emulsion or suspension or salve.

Thus, the therapeutic compositions may be formulated for parenteral administration (e.g., by injection, bolus injection or continuous infusion) and may be presented in unit dose form in ampules, pre-filled syringes, small volume infusion containers or in multi-dose containers. As noted above, preservatives can be added to help maintain the shelve life of the dosage form. The active compositions and other ingredients may form suspensions, solutions, or emulsions in oily or aqueous vehicles, and may contain formulatory agents such as suspending, stabilizing and/or dispersing agents. Alternatively, the active compositions and other ingredients may be in powder form, obtained by aseptic isolation of sterile solid or by lyophilization from solution for constitution with a suitable vehicle, e.g., sterile, pyrogen-free water before use.

These formulations can contain pharmaceutically acceptable carriers, vehicles and adjuvants that are well known in the art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol,” polyglycols and polyethylene glycols, C₁-C₄ alkyl esters of short-chain acids, ethyl or isopropyl lactate, fatty acid triglycerides such as the products marketed under the name “Miglyol,” isopropyl myristate, animal, mineral and vegetable oils and polysiloxanes.

It is possible to add, if necessary, an adjuvant chosen from antioxidants, surfactants, other preservatives, film-forming, keratolytic or comedolytic agents, perfumes, flavorings and colorings. Antioxidants such as t-butylhydroquinone, butylated hydroxyanisole, butylated hydroxytoluene and α-tocopherol and its derivatives can be added.

Additionally, the compositions are well suited to formulation as sustained release dosage forms and the like. The formulations can be so constituted that they release the active composition, for example, in a particular part of the intestinal or respiratory tract, possibly over a period of time. Coatings, envelopes, and protective matrices may be made, for example, from polymeric substances, such as polylactide-glycolates, liposomes, microemulsions, microparticles, nanoparticles, or waxes. These coatings, envelopes, and protective matrices can be used to coat indwelling devices, e.g., stents, catheters, peritoneal dialysis tubing, draining devices and the like.

The pharmaceutical formulations of the present invention may include, as optional ingredients, pharmaceutically acceptable carriers, diluents, solubilizing or emulsifying agents, and salts of the type that are available in the art. Examples of such substances include normal saline solutions such as physiologically buffered saline solutions and water. Specific non-limiting examples of the carriers and/or diluents that are useful in the pharmaceutical formulations of the present invention include water and physiologically acceptable buffered saline solutions such as phosphate buffered saline solutions pH 7.0-8.0.

Through incorporation of the n-3 HUFA fatty acid compositions provided herein into foods, long-term ingestion of the foods can be achieved, advantageously leading to attainment of the effects of preventing and treating life-style related diseases. The food of the present invention may include, in addition to general food products, foods for promoting health through specific functions, such as health foods, functional foods, and foods for specified health use. In order to prepare these specific foods, the food of the present invention may be formed into tablets, granules, powders, etc., rather than as food products. Examples of the food products include bakery-related products such as breads, cakes, cookies, pies, pizza crusts, and bakery mixtures; O/W type oil/fat processed products such as dressings, mayonnaise sauces, coffee creamers, and whipped creams; W/O type oil/fat processed products such as margarines, spreads, and butter creams; snack foods such as chocolates, potato chips, ice creams, and desserts; milk products such as milk, cheese, and yogurt; beverages; sauces; liquid seasonings for grilled meat; peanut butter; shortenings for frying and baking; processed meats such as hams, sausages, and hamburgers; noodles; frozen foods; retort-pouched foods; and cooking oils for deep-frying such as fries, tempura, and the like, as well as frizzling. These food products are prepared from the oil/fat composition of the present invention and typical food raw materials in accordance with the target food product. Since the oil/fat composition of the invention can be incorporated into a variety of foods, the composition can be ingested daily without any special effort. Generally, the oil/fat composition of the present invention is incorporated into a food in an amount, which varies in accordance with the type of the food, of preferably 0.05-100%, particularly preferably 0.5-80%.

Furthermore, the active ingredients may also be used in combination with other therapeutic agents, for example, agents for the treatment of cardiovascular disorders, pain relievers, anti-inflammatory agents, antihistamines, bronchodilators and the like, agents for the treatment of obesity, agents for the treatment of hypertension, agents for the treatment of diabetes, whether for the conditions described or some other condition.

For example, in certain embodiments the n-3 HUFA fatty acid compositions provided herein are administered in conjunction with a 3-hydroxy-3-methylglutaryl coenzyme A reductase inhibitor. Suitable exemplary 3-hydroxy-3-methylglutaryl coenzyme A reductase inhibitor include simvastatin, atorvastatin, mevastatin, lovastatin, pravastatin, fluvastatin, rosuvastatin, itavastatin, and visastatin.

In certain other embodiments, the n-3 HUFA fatty acid compositions provided herein are administered in conjunction with a drug or composition selected from neuroleptic antipsychotics tricyclic antidepressants, selective serotinin reuptake inhibitors, antiepilileptics, and lithium drugs.

In certain other embodiments, the n-3 HUFA fatty acid compositions provided herein are administered in conjunction with antihypertensive agents. Suitable exemplary antihypertensive agents may include diuretics, β₁-selective adrenergic antagonists (sometimes referred to as “beta-blockers”), calcium channel blockers, angiotensin-converting enzyme (“ACE”) inhibitors, angiotensin II receptor antagonists (sometimes referred to as “angiotensin receptor blockers” or “ARBs”), or alpha-1 receptor blockers (sometimes referred to as “alpha-blockers”). Vasodilator medications such as hydralazine, minoxidil, diazoxide, or nitroprusside may be required if the blood pressure is very high. Diuretics, sometimes called “water pills,” flush water and salt out through the urine. Diuretics are often the first high blood pressure medications prescribed and include, for example, thiazides such as chlorthalidone, furosemide, hydrochlorothiazide, and spironolactone. β₁-selective adrenergic antagonists target receptors in the heart and blood vessels, making the heart pump at a slower rate and with less force. Beta-blockers include acebutolol, arvedilol, atenolol, betaxolol, bisoprolol, bopindolol, bucindolol, carteolol, carvedilol, celiprolol, esmolol, labetalol, levobunolol, medroxalol, metipranolol, metoprolol, nadolol, nebivolol, oxprenolol, penbutolol, pindolol, propafenone, propranolol, sotalol, and timolol maleate. Calcium channel blockers help control the flow of calcium into cells, which helps the heart and blood vessels to relax. Calcium channel blockers include, for example, nisoldipine, verapamil, diltiazem, nifedipine, nimodipine, felodipine, nicardipine, isradipine, amlodipine, and bepridil. Angiotensin converting enzyme (ACE) inhibitors prevent the formation of angiotensin II, the hormone that causes blood vessels to contract. ACE inhibitors cause the blood vessels to relax thereby lowering blood pressure. ACE inhibitors include, for example, captopriol, fentiapril, pivalopril, zofenopril, alacepril, enalapril, enalaprilat, enalaprilo, lisinopril, benazepril, quinapril, and moexipril. Angiotensin II receptor antagonists include, for example, losartan, candesartan, irbesartan, valsartan, telmisartan, eprosartan, and olmesartan medoxomil. Alpha-1 receptor blockers control nerve impulses, allowing blood vessels to relax and blood to flow without encountering as much pressure. Instead of preventing the formation of angiotensin II, angiotensin receptor blockers, block the blood vessels from angiotensin II. Alpha-1 receptor blockers include, for example, doxazosin, terazosin, and prazosin. Vasodilators include, for example, hydralazine, Minoxidil, sodium nitroprusside, isosorbide dinitrate, and diazoxide, as well as bosentan, eporprostenol, treprostinil, and iloprost. Other less frequently used hypertension medications include α-adrenergic receptor antagonists (e.g., prazosin, terazosin, doxazosin, ketanserin, indoramin, urapidil, clonideine, guanabenz, guanfacine, guanadrel, reserpine, and metyrosine), sympatholytic agents (e.g., methyldopa), ganglionic blocking agents (e.g., mecamylamine and trimethaphan), and endothelin receptor antagonists (e.g., bosentan and sitaxsentan).

In certain other embodiments, the n-3 HUFA fatty acid compositions provided herein are administered in conjunction with antiobesity agents. Suitable exemplary antiobesity agents may include those that lower body fat and include, for example, appetite suppressants, anorectics (including phentermine, mazindol, diethylpropion, and phendimetrazine), lipase inhibitors (including orlistat), exendins and exendin agonists (including exendin-4), amylins and amylin agonists (including pramlinitide), leptins, GLP-1 and GLP-1 agonists (including Arg(34)Lys(26)-(N-ε-(γ-Glu(N-α-hexadecanoyl))-GLP-1(7-37), sometimes referred to herein as GLP-1LA)), and adrenergic receptor agonists (including sibutramine).

In certain other embodiments, the n-3 HUFA fatty acid compositions provided herein are administered in conjunction with insulin and insulin like agents. Suitable exemplary insulins and insulin like compounds may include (1) rapid-acting insulins (also sometimes referred to as “monomeric insulin analogs”); (2) short-acting insulins (also sometimes referred to as “regular” insulins); (3) intermediate-acting insulins; (4) long-acting (also sometimes referred to as “basal insulins”); (5) ultra-long acting insulins, (6) pI-shifted insulin analogs; (7) insulin deletion analogs; (8) derivatized insulins; (9) derivatized insulin analogs; (10) derivatized proinsulins; (11) human insulin analog complexes (e.g., hexamer complexes), (12) insulin mixtures, and (13) PEG-insulins. Insulins may be present in the compositions of the invention an amount, for example, that is effective to (1) lower blood glucose, (2) lower serum glucose, (3) lower urine glucose, (4) lower glycosylated hemoglobin (HbA_{1c}) levels, (5) lower fructosamine, (6) lower postprandial glycemia, (7) ameliorate impaired glucose tolerance, (8) ameliorate impaired fasting glucose, and/or (9) lower the rate and/or severity of hypoglycemic events, including severe-hypoglycemic events.

In certain other embodiments, the n-3 HUFA fatty acid compositions provided herein are administered in conjunction with antihypoglycemic agents. Suitable exemplary antihypoglycemic agents may include biguanides (for example, metformin), thiazolidinediones (for example, troglitazone, rosiglitazone, and pioglitazone), α-glucosidase inhibitors (for example, acarbose and miglitol), and sulfonylureas (for example, tolbutamide, chlorpropamide, gliclazide, glibenclamide, glipizide, and glimepiride). Such compounds may be present in the compositions of the invention an amount, for example, that is effective to (1) lower blood glucose, (2) lower serum glucose, (3) lower urine glucose, (4) lower glycosylated hemoglobin (HbA_{1c}) levels, (5) lower fructoseamine, (6) lower postprandial glycemia, (7) ameliorate impaired glucose tolerance, (8) ameliorate impaired fasting glucose, and/or (9) lower the rate and/or severity of hypoglycemic events, including severe hypoglycemic events.

In certain other embodiments, the n-3 HUFA fatty acid compositions provided herein are administered in conjunction with agents directed to neurodegenerative diseases or disorder or conditions. Exemplary disorders include Huntington's disease, Parkinson's disease, Alzheimer's disease, Schizophrenia, Major Depression Unipolar, Bipolar depression, Obsessive compulsive disorder, borderline personality disorder, Post natal depression, Organic Brain damage, and Traumatic brain injury. Suitable exemplary agents for such coadministration with compositions of the invention may include Levodopa drugs, such as, for example, levodopa; and levodopa / carbidopa; dopamine agonists, such as, for example, apomorphine, ropinirole, pramipexole and cabergoline, pergolide, bromocriptine, talipexole, and lisuride; Anticholinergics, such as, for example, trihexyphenidyl, orphenadrine, benztropine mesylate procyclidine hydrochloride, diphenhydramine, and ethopropazine, benzhexol, trihexyphenidyl, benztropine, anisotropine, atropine, belladonna, clidinium, dicyclomine, glycopyrrolate, homatropine, hyoscyamine, mepenzolate, methantheline, propantheline, and scopolamine; comt inhibitors, such as tolcapone and entacapone; anti-viral drug, such as, for example, amantadine hydrochloride; and mao inhibitors, such as, for example, isocarboxazid, phenelzine, tranylcypromine and selegiline hydrochloride; levetiracetam; antihistamines, such as, for example, diphenhydramine, hydroxyzine; tiapiride, pimozide, haloperidol, tertrabenazone and phenothiazines, including, but not limited to, chlorpromazine, fluphenazine, mesoridazine, perphenazine, prochlorperazine, thioridazine, trifluoperazine, and triflupromazine; drugs use to treat behavioral symptoms, such as, for example, depression, anxiety, irritability, aggression and psychosis; tricyclic antidepressants, such as, for example, amitriptyline, imipramine, desipramine, nortriptyline, amoxapine, clomipramine, desipramine, doxepin, protriptyline, trimipramine; selective serotonin reuptake inhibitors (ssris), such as, for example, fluoxetine, fluvoxamine, paroxetine, sertraline, and citalopram escitalopram oxalate; mao inhibitors, such as, for example, isocarboxazid, phenelzine, tranylcypromine and selegiline hydrochloride; benzodiazepines: alprazolam, chlordiazepoxide, clonazepam, clorazepate, diazepam, estazolam, flurazepam, halazepam, lorazepam, oxazepam, quazepam, temazepam, and triazolam; classical antipsychotics, such as, for example, haloperidol, thiothixine, and loxapine; anti-psychotics, such as, for example, clozapine, risperidone, olanzapine, quetiapine, and ziprasidone, and aripiprazole; drugs to treat hypokinesia; Miraxion; Coenzyme Q10; Riluzole; ethyl eicosapentaenoate (ethyl EPA); agents directed to amyotrophic lateral sclerosis (als); anti-spastic, such as, for example, lioresal (Baclofen); amnestic, anti-anxiety, anticonvulsant, anti-tremor, skeletal muscle relaxant: such as, for example, benzodiazepines, including alprazolam, chlordiazepoxide, clonazepam, clorazepate, diazepam, estazolam, flurazepam, halazepam, lorazepamn, oxazepam, quazepam, temazepam, triazolam, anti-dyskinetics, such as, for example, trihexyphenidyl; amitriptyline, including Anafranil, Asendin, Aventyl, Elavil, Endep, Norfranil, Norpramin, Pamelor, Sinequan, Surmontil, Tipramine, Tofranil, Tofranil-PM, Vivactil; Ceftriaxone; AVP-923-Neurodex; IGF-1 (Myotrophin™); inocycline; Buspirone; Creatine; TCH346, Ritonavir and hydroxyurea combination; AEOL 10150; Sodium phenylbutyrate; Celebrex; NeotrofinT (AIT-082, leteprinim potassium); NAALADase (N-Acetylated-Alpha-Linked-Acidic-Dipeptidase); Oxandrolone; Topiramate (Topamax); Xaliproden; Indinavir, (Tysabri®); interferon beta-1a (Avonex(®); interferon beta-1a (Rebif®); glatiramer acetate injection (Copaxone); mitoxantrone (Novantrone); anti-depressants; betamethasone, cortisone, dexamethasone, hydrocortisone, methylprednisolone, prednisolone, prednisone and triamcinolone; Benodiazepines; lioresal (Baclofen); tizanidine (Zanaflex); Amanitadine Symmetrel; ABT-874; Inosine; RG2077 (CTLA4-IgG4m); daclizumab (Zenapax®); Donepezil (Aricept®) and glucose; BHT-3009 Rituxan (rituximab); AVP-923; Betaseran/Copaxone; RO0506997; Cholinesterase Inhibitors, such as, for example, Donepezil (Aricept®); Rivastigmine tartrate (Exelon®); Galantamine HBr (Razadyne®, which was formally Reminyl®); Tacrine (Cognex);Memantine hydrochloride (Namenda®); NeotrofinT (AIT-082, leteprinim potassium); Xaliproden; PPI-1019; Neramexane; SGS742; Transdermal 17-β-estradiol; and Valproate.

The invention is further illustrated by the following non-limiting Examples.

### EXAMPLE 1

### EPA Composition

This Example describes the making of a composition produced by a heterotrophic or primarily heterotrophic batch culture of a microorganism to produce a microbial biomass process containing substantially only EPA in its n-3 HUFA fraction and relatively low levels of C14:0 and C16:0. The microorganism used is N laevis.

Culture Origin and Preculture Conditions.

The diatom N. laevis (UTEX 2047) was obtained from the University of Texas Culture Collection of Algae. Axenic precultures were grown heterotrophically as batch cultures in 500 ml Erlenmeyer flasks in a basal medium comprising Lewin's Diatom Medium modified with 15 mg per liter Na2SiO39H2O, and adjusted to comprise a total of 600 mg per liter of sodium nitrate. Dextrose (100% glucose) was added to the media prior to autoclaving to make up a concentration of 5 grams per liter of glucose. Flasks containing the precultures were placed on an orbital shaker at 150 rpm in a dark room at 25 DEG C. for 1 week prior to the experiment.

Culture Media.

A modified Lewin's Diatom Medium (mLDM) is prepared from locally available ingredients according to the recipe of the UTEX website (presently www.bio.utexas.edu/research/utex). 15 mg/L of sodium metasilicate 9H2O is added o the medium together with sufficient sodium nitrate to achieve a total concentration of sodium nitrate of 600 mg per liter. The mLDM was adjusted to pH 8.5 by titration of a dilute base solution such as titration of 0.1 molar strength potassium hydroxide, autoclaved at 121 deg Celsius for 15 minutes and then rapidly cooled to prevent formation of precipitates. Four pure sugars; glucose (G), sucrose (S), D(−) fructose (F) and lactose (L) and well as six combinations of paired sugars in equal amounts (GS, GF, GL, SF, SL, FL) were added to media to produce batches with a final concentration of 5 g 1-1 of each sugar or paired sugar combination. All experiments were carried out in triplicate. Flasks were placed in a dark room at 25 DEG C. and bubbled with 0.2 micron filtered air to maintain circulation in the culture for 11-12 days. On day 12 biomass was harvested from all remaining flasks by repeat centrifugation at 2500 g followed by withdrawal of media and replenishment with distilled water. Samples were dried under a stream of nitrogen and lipids were extracted and methylated in a one-step incubation procedure with a mixture of toluene, methanol and sulphuric acid. The mixture was neutralized, and the extract purified and dried with charcoal and anhydrous sodium sulphate. Fatty acids are determined by comparison with a standard injected into the sample before extraction. An aliquot of solute from a sample dissolved in hexane was then taken and analysed by Gas Chromatograph (GCFID 6890; Agilent Corp) with a 105 meter 0.25 mm 90% bis-cyanoprofile column (RIX2330; Restek) and 0.25 micron film thickness. Fatty acids were identified by retention time in comparison with a 37-component mixture (Suplco). Peak areas are then corrected for peak response using theoretical response factors (AOCS). and results were reported as area under the curve. (Sukhija, P. S. & Palmquist, D. L. (1988) Rapid method for determination of total fatty acid content and composition of feedstuffs and feces. J. Agric. Food Chem. 36: 1202-1206. and Wu, J., James, Jr. D. W., Dooner, H. K. & Browse, J. A mutant of Arabidpsis deficient in the elongation of palmitic acid. Plant Physiol. 106: 143-150.). (1994). When biomass harvested from culture flasks were grouped into those containing at least a proportion of one individual sugar. The following results were obtained.
Percentage EPA in total fatty acids of biomass.
   Fructose: 14.06 S.D.=2.17
   Lactose: 13.71 S.D.=1.17
   Sucrose: 13.62 S.D.=1.42
   Glucose 12.27 S.D.=1.35
Percentage C16;0 in total fatty acids of biomass.
   Fructose: 21.62 S.D. 0.54
   Lactose: 22.52 S.D. 1.33
   Sucrose: 22.90 S.D. 1.36
   Glucose 23.50 S.D. 0.88
Total fatty acid production per flask in milligrams.
   Fructose: 0.77, S.D.=0.42
   Lactose: 0.67, S.D.=0.27
   Sucrose: 0.65, S.D.=0.32
   Glucose: 0.44, S.D.=0.25

The results of work reported in this example (above) suggests that those culture media containing at least a proportion of those sugars capable of producing relatively high yields of total fatty acid from the microalgal species Nitzschia laevis were simultaneously able to produce able to produce compositions with higher EPA and lower C16:0 that those culture media containing at least a proportion of sugars producing relatively lower yields of total fatty acid.

Thus when flasks containing culture media with at least a proportion of fructose were grouped together for analysis it could be shown that the resultant biomass simultaneously produced a higher total fatty acid yield, together with a higher proportion of EPA and a lower proportion of C16:0 in total fatty acids compared to all other flasks grouped according to content of other sugars. Those containing at least a proportion of lactose, had the second highest yield of fatty acids, the second highest proportion of EPA in fatty acids and the second lowest proportion of C16:0, and sucrose the third highest yield of fatty acids, the third highest proportion of EPA in fatty acids and the third lowest proportion of C16:0. When grouped together biomass produced in flasks containing culture media with at least a proportion of glucose had the lowest yield of fatty acids, coupled with the lowest amount of EPA and the highest percentage of C16:0 as a proportion of total fatty acids.

This suggests that glucose alone is not the preferred carbon source for production of EPAOILs with relatively low C16:0 from Nitzschia laevis as compared to culture media containing at least a proportion of fructose or lactose or sucrose.

### EXAMPLE 2

### EPA Composition

This Example describes the making of a composition produced by a heterotrophic or primarily heterotrophic batch culture of a microorganism to produce a microbial biomass process containing substantially only EPA in its n-3 HUFA fraction and relatively low levels of C14:0 and C16:0. The microorganism used is N laevis.

A modified Lewin's Diatom Medium (mLDM) was prepared from locally available ingredients according to the recipe of the UTEX website presently www.bio.utexas.edu/research/utex). 15 mg/L of sodium metasilicate 9H2O was added to the medium together with sufficient sodium nitrate to achieve a total concentration of sodium nitrate of 600 mg per liter. The mLDM is adjusted to pH 8.5 by titration of a dilute base. solution such as titration of 0.1 molar strength potassium hydroxide, autoclaved at 121 deg Celsius for 15 minutes and then rapidly cooled to prevent formation of precipitates.

Concentrated solutions of either D(−)fructose, glucose, lactose or sucrose were added aseptically through a syringe filter to achieve a concentration of 2.5 grams of one of the sugars in the medium. Twelve 500 ml Ehrlenmeyer flasks were filled in an aseptic environment with 200 ml of the culture medium and inoculated by sterile loop with cells from a slope containing viable cells of N laevis (UTEX 2047) obtained from the University of Texas Micro-algal Culture Collection. Cells were precultured by bubbling sterile air through the flask in total darkness for seven days before being placed under one of three different very low intensity light conditions characterized by different wavelengths of light. Constant light comprising wavelengths either spread relatively evenly throughout the visible spectrum of 360-720 nanometers (white) or restricted predominantly to the 450-600 nanometer (blue) or 600-760 nannometer (red) by gel filtration was provided by twin white Flourescent lamps (Phillips). Light intensity was adjusted by digital dimming balasts to a photon flux density of 2.5 micromol photons per square meter per second as measured at the base of the flasks by a quantum sensor (Apogee QSO-ELEC) and supplied continuously for a further seven days. On day 14 cells are harvested by centrifugation at 3000 rpm for two minutes and stored frozen at minus 20 DEG C. prior to fatty acid analysis. After thawing fatty acids in biomass samples were extracted and analyzed by the method described in Example 1a.

Biomass harvested from culture flasks was grouped into those exposed to the same light conditions to reveal that white light and red light produced higher fatty acid yields (0.743 S.D.=0.513 and 0.762 S.D.=0.600 respectively) than blue light (0.596 S.D.=0.342).

The mean EPA and C16:0 contents across all flasks any each light condition was highest under white light (15.522 S.D.=2.454 and 20.618 S.D.=1.410 respectively) followed by blue (15.214 S.D.=1.332 and 20.514 S.D.=0.689 respectively) and then red (11.894 S.D.=1.352 and 21.699 S.D.=0.637 respectively). P-values were calculated by a two tailed students T-test. The reported differences between EPA and C16:0 percentages in biomass from flasks exposed to red versus blue light were considered to be statistically significant with P-values of 0.045 for C16:0 and 0.013 for EPA. There was also an apparent consistent trend towards higher EPA and lower C16:0 across the three light conditions with white light being preferred over blue and red light returning the least preferred compositions.

Across all light conditions mean lipid yields for the lactose, fructose, sucrose and glucose were as follows (1.073 S.D.=0.522; 0.778 S.D.=0.240; 0.644 S.D.=0.609; 0.274 S.D.=0.049). Only fructose was significant in terms of fatty acid yields when compared to glucose the difference having a P-values of 0.023. The difference between lactose and glucose fatty acid yields approached significance with a P-value of 0.058.

No pattern was observed and no significant differences were measured between percentages of EPA and C16:0 when biomass produced via exposure to different light conditions was grouped according to sugars in the media.

The best biomass fatty acid composition was obtained from a flask containing lactose in the culture medium and grown under low intensity white light. This is reported as follows in percentage fatty acids
C20:5=18.21
C16:0=19.3
C14:0=7.75
C22:6=2.97

This culture condition also produced a yield of fatty acids of 1.254, which was the almost the highest recorded second only to lactose under red light.

The results of work reported in this example suggest that those culture media containing fructose and possibly lactose may be suitable for attaining higher fatty acid productivities from batch cultures of the microalgal species Nitzschia laevis produced under low intensity light. A preferred EPAOIL composition was produced via the use of lactose as the sole sugar source under white light.

While only the differences between EPA and C16:0 in biomass between red and blue conditions was considered to be statistically significant an apparent trend was observed towards higher EPA and lower C16:0 across the three light conditions with white light being preferred over blue and red light returning the least preferred compositions. This suggests that red or white light is the preferred low intensity light source for the production of EPAOILS with relatively low C14:0 and C16:0 levels.

### EXAMPLE 3

### EPAOIL Production

This Example provides various products comprising compositions rich in EPA which can be produced in sufficient volumes such that they are suitable for human or animal ingestion in the first instance. Production is most feasible by means of cultivation of a micro-alga. If the set of genes providing for synthesis of EPA were to be taken from an organism possessing same, and transferred into another organism, such as a plant or animal, along with gene control mechanisms providing for expression of said genes at a reasonable level, then it might no longer be necessary to rely on micro-algae as the original source of EPA.

This Example includes human-acceptable food ingredients (such as spreads, powders, flavouring, vegetable fat-like materials, or oils not of pharmaceutical purity—20-50% EPA), functional foods (such as prepared chocolate bars, energy bars, dehydrated foods, or the like), beverages, dietary supplements, by which we mean products that are sold over the counter as remedies for perceived or actual conditions or sub-optimal health, and nutraceuticals by which we mean foodstuffs that may be prescribed for the alleviation or treatment of various disorders, or to maintain wellbeing; and animal-acceptable supplements (such as pellets, extrusions, suspensions, drenches, sprays).

A preferred embodiment of the present invention involves the provision of compositions comprising or derived from the whole cell EPA-rich biomass of the micro-alga N. laevis (UTEX 2047). Alternatively other microorganisms may be used capable of being produced heterotrophically or primarily heterotrophically. Though there may be natural species of micro-algae capable of yet higher yields of EPAOILS which are the same time have relatively low levels of C14:0 and C16:0 when cultivated the great amount of published work has not yet located those species.

Biomass produced via the method of Example 2 can be harvested by simple sedimentation and the medium decanted. Sedimented cells may be collected and centrifuged at 3000 rpm for two minutes. The resulting biomass can then be used to inoculate a reactor of 3 liters in size agitated by a Ruston-type impellor. By simple adjustment of flask size, and aeration method, both techniques well known to those skilled in the art, this will provide for large volumes of inocula which can be used to inoculate larger vessels.

One or several bioreactors of 20-200,000 litres using impellors or an air lift and geometric design to regulate hydrodynamic turbulence and gas mass transfer is employed to produce sufficient volumes of N. laevis biomass for processing into supplements suitable for incorporation into the diets of non-aquaculture food producing animals. Design parameters for such reactors are well known by those skilled in the art and can be easily arrived by reference to texts such as Chisti M. Y., Airlift Bioreactors Elsevier Applied Science 1989 and M. J. Kennedy, A review of the design of reaction vessels for the submerged culture of micro-organisms. Industrial Processing Division, Dept. of Scientific and Industrial Research, 1984.

One or a number of probes capable of monitoring pH, dissolved oxygen, dissolved carbon di-oxide, salinity, selective ions, temperature and optical density or turbidity can be inserted into the reactor and signals relayed if necessary through intermediate transmitters to a programmed logic controller capable of feeding back control signals to process control apparatus in order to dynamically alter said environmental variables in the reactor. Such process control apparatus may include but are not limited to pumps including diaphram and peristaltic pumps, electrically actuated valves, electric motors, compressors, electric heating elements centrifuges, magnetically operated chambers, ultrasound apparatus, and electric lights. Temperature is maintained at 20 deg C. via the use of warming blankets, jacketed reactors or heat exchange coils all methods well known to those of skill in the art and which may be regulated via current limiting devices and if necessary via pumped media Dissolved oxygen and carbon dioxide levels, are controlled, by injection of sterile air, pressure regulation of the reactor, and adjustment of motor speed in the case of a reactor agitated with a motor driven impellor . A preferred concentration for dissolved oxygen is about 3 parts per million. Optimal carbon dioxide levels may be arrived at by methods well known to those skilled in the art. pH is kept at around 8.0 via injection of either potassium hydroxide or dilute hydrochloric acid through dynamically controlled peristaltic pumps. Cell density is estimated from turbidity or optical density measurements and controlled via a cell recycling system. Such cell recycling systems are well known to those of skill in the art. Reference to Z-Y Wen and S F Chen in Process Biochemistry 38: (2002) 523-529 might prove useful. Such strategies involve replacing growth medium whilst limiting the extent to which cells are lost from the medium. This has the advantage of removing substances that inhibit growth from the medium resulting in continued growth at high cell densities.

Alternatively the medium may be treated by mechanical, chemical or other physical means so as to remove antialgal or autoinhibitory metabolites that inhibit growth or productivity of the cells.

Alternatively, cells may be treated during the process by mechanical, chemical or other physical means so as to alter the production of secondary metabolites which may otherwise inhibit growth and/or production of EPA.

Culture media is to comprise a basal media of modified LDM medium as described in Example 1.

Alternatively culture media may comprise natural seawater or dilute natural seawater.

Alternative basal media may be arrived at by those with skill in the art.

In a preferred embodiment the culture media may contain low levels of chlorides to reduce the level of likely corrosion in stainless steel reaction vessels.

A preferred culture medium has a sugar mixture containing at least a part lactose or fructose added to it aseptically such that the total sugar content is equivalent to 5 or 20 grams or more per liter.

Alternatively organic carbon sources are to contain at least a part of these sugars an alternative method might utilize media containing only other individual sugars or mixtures thereof (e.g. glucose, sucrose maltose) or more complex carbon sources such as starch or sugar-containtaining industrial byproducts such as whey streams from the dairy industry. Alternatively lower concentrations of sugars may also be utilized. Nitrogen in a form assimiliable to the microorganism is supplied to the culture in culture medium or stock solutions such that the total available nitrogen ranges from 30 mg per liter to 3 grams per liter. A preferred source of nitrogen is sodium nitrate. Alternatively more complex nitrogen sources may be used including but not limited to yeast extract tyrptone, corn starch liquor etc.

Silicate is supplied to the culture medium such that concentrations of silicate are between about 15 mg and 200 mg per liter. A preferred source of silicate is sodium metasilicate in ether the 5 or the 9 hydrate form.

Culture medium is to be replaced constantly via dilution. In cultures with high nutrient content dilution rates employed may be lower than those with high nutrient concentrations. A preferred dilution rate for cultures with a sugar content of 20 grams or more per liter is about (>0.4).

Alternatively the micro-algal cells may be exposed to light varying in intensity, frequency and duration in order to enhance production efficiency and/or evoke a physiological response commensurate with improving the composition of the biomass.

A preferred method involves exposing cells in a medium containing lactose as the sole sugar source to very low total amounts of white light. This may be achieved via continuous illumination with a light intensity providing a total light exposure to each cell equivalent to that which would be provided by a white fluorescent light of a photon flux density of 2.5 micromol photons per square meter per second measured at the base of well mixed 500 ml Erlhenmeyer flask containing 200 ml of a dilute cell suspension of about 1 gram or less of microalgal biomass per liter. Total per cell light exposure may be calculated by a number of methods well known in the art (See: Grima, E. M., Fernández, F. G. A., Camacho, F. G., and Chisti, Y., J. Biotechnol., 70, 231-247 (1999). Photobioreactors: light regime, mass transfer, and scaleup.)

Alternatively a spectrum of light can be utilized employing filtered lights, such that wavelengths are concentrated either predominantly in the 400-500 or 600-800 nanometer range or a mixture thereof.

Alternatively, the supply of nutrients may be altered during particular stages of the process with or without illumination in one or more “finishing steps” where nutrient limitation or alteration of nutrient ratios is employed so as to increase production efficiency or improve the composition of resultant biomass. Such strategies are well known to those of skill in the art and may include alteration of the availability of nitrogen and silicate or alteration of carbon nitrogen ratios.

Alternatively the process may be operated in a continuous mode, by a) extracting fatty acids from a sample of the biomass and analyzing fatty acid composition by the methods of Example 1 and then b) adjusting the above culture parameters it should be possible to obtain large quantities of biomass comprising preferred compositions. It is preferred that the said microalgal biomass will comprise more than about 18% C20:5 n-3, less than about 20% C16:0, less than about 11% C 14:0, at least about 0.1% C18:3 n-3 and an EPA to DHA ratio of bout 6:1 or more.

Cells harvested during or at the termination of the culture may tend to form aggregates and settle out easily or the process of sedimentation may be assisted by flocculation. The following reference may be useful; Boonaert et al., “Cell separation, Flocculation” Encylopedia of Bioprocess Technology Fermentation, Biocatalysis (1999) and Bioseparation Volume 1: 531-47. Alternatively the technique given by M. E. Cartens et al., in J Am Oil Chem Soc 73: 1025-1031 may be followed.

Water may be removed from the harvested cells by filtration and/or centrifugation then further dried by drum, spray dried or freeze drying. These techniques are well know to those skilled in the art but the following reference may be useful; Letki, A. G. “Know when to turn to centrifugal separation”, Chem Eng. Prog., September 29-44 (1998); Bruttini R. “Freeze drying, pharmaceuticals”, Encylopedia of Bioprocess Technology Fermentation, Biocatalysis and Bioseparation 2: 1276-98 (1999)

Dry or semi dry biomass may be stored in refrigerated vats as a slurry extruded or formed into cakes, pellets or powders. These may then be formulated into food products for human or animal consumption. Total lipids can be extracted from dried or wet biomass by extraction using volatile organic solvents such as ethanol or hexane or supercritical gas chromatography. These methods which are well known to those skilled in the art may also be employed to concentrate extracted lipids to produce intermediate or high purity EPA oil fractions.

Useful methods are given by Cartens M. E. et al., in “Eicosapentaenoic Acid (20:5 n-3) from the Marine Microalga Phaeodactylum tricornutum” J Am Oil Chem Soc 73: 1025-1031 (1996); Ibanez M. J. et al., “Optimization of fatty acid extraction from Phaeodactylum UTEX 640 biomass” J Am Oil Chem Soc 75: (12) 1735-1740 (1998); Walker, “Supercritical CO2 extraction of Lipids from Pythium irregulare” J Am Oil Chem Soc 76: 595-602 (1999). Optimization of supercritical CO2 may be made in light of Yener “Estimation of lipid properties related to supercritical fluid extraction” Int J Food Properties 4:1 45-57 (2001) which leads towards separation of types of lipid.

Bulk extracted lipids may be processed further immediately or stored prior to further processing. To prevent oxidation of fatty acids during storage antioxidants may be added, oil may be cooled, protected from exposure to oxygen by sparging with an inert gas such as nitrogen or argon, protected from light by dark storage. For lipids extracted using hexane the following reference may be of use to practitioners: Guil-Guerrerro J. L., “Hexane reduces peroxidation of fatty acids during storage” Eur J Lipid Sci Technol 103: 271-278 (2001).

Oils may be further processed into powders, gels, microencapsulated or soft gel encapsulated, blended with existing foods, food ingredients or neutraceuticals, further processed to alter colour, flavor and other sensory characteristics.

### EXAMPLE 4

### Production of EPA Foods

This Example describes the production of EPA foods by feeding supplements containing substantially only EPA as their n-3 HUFA component and relatively low or nondetectable levels of C14:0 and C16:0 to non-aquaculture food producing animals. The administration of compositions containing substantially only EPA as their n-3 HUFA component and relatively low or non-detectable levels of C14:0 and C16:0 to non-aquaculture food-producing animals and the resulting production of novel food products with desirable modified fatty acid profiles is described.

Another way to pre-process compositions for preparation of human food is to pass the biomass through an existing type of food-producing animal, essentially adding a link to the food chain. The food products derived should be just as acceptable as traditional foods and food products from the food-producing animal, with the benefit that the fatty acid profile is advantageous to the health of the human. Although there is inherent inefficiency in the introduction of extra links, surprising advantages are present.

Typical products included under Example 2 are: those based on consumption of secretions or of body parts of animals (such as milk meats, offals, eggs and other organs or tissues). Cows and other bovine animals, sheep, goats, deer, pigs, horses and camellids (including camels, alpacas, and the like) are included (without limitation) within the group of milks and/or meat meat-producing manmmals. Lard (shortening) and other “pure-fat.” products are noteworthy of capable of being enhanced with defined n-3 HUFAs. Chickens, turkeys and many other species of farrned birds together provide a substantial proportion of human intake of meats.

Recent medical evidence suggests dietary supplementation with the omega-3 fatty acid EPA may be more beneficial for many individuals than supplementation with its counterpart DHA. See: Horrobin, D. F., Prog Drug Res; 59: 171-99 (2002). Both these fatty acids are present in low levels or are undetectable in cows milk, but are present in fairly high concentrations in many naturally occurring oils of marine origin. Fortified “functional” foods including milk products are increasingly becoming utilized which have been produced via supplementation of the diets of food producing animals with fish oils rich in both EPA and DHA and with microalgae rich in DHA. In the present example a milk is produced containing EPA but substantially no DHA in its fatty acid component via supplementation of the diets of lactating bovines with a supplement containing substantially only EPA.

Plants, the normal dietary component for grazing animals such as cows, do not naturally produce highly unsaturated omega-3 fatty acids (n-3 HUFA). Thus, it is not surprising that the extent to which bovines are able to convert alpha linolenic acid (ALA), the most common metabolic precursor of n-3 HUFA found in plants, into n-3HUFA appears to be limited. Hauswirth et al. “Report milkfat from grass fed cows in alpine areas contains higher levels of both alpha linolenic acid (ALA) and of the n-3 HUFA eicosapentaenoic acid (EPA) as well as lower levels of palmitic acid than milkfat of cows in non-alpine areas”, Circulation, 109: 103-107 (2004). Petit et al. (2002b) however, report that duodenal infusion of 500 grams per day of ALA led to a very high level of ALA in milkfat (14%) with no concomitant increase in EPA (Milk Production and Composition, Ovarian Function, and Prostaglandin Secretion of Dairy Cows Fed Omega-3 Fats1 Dairy Sci. 85: 889-899 American Dairy Science Association, (2002)). Without recourse to post harvest fortification therefore in order to produce milkfat with significantly raised n-3 HUFA levels it is necessary to supplement the diets of bovines with preformed n-3 HUFA.

When compositions containing both EPA and DHA are fed to lactating dairy cows however, the concentrations of both fatty acids increase in milk. Thus in order to produce EPA-only milk it may be necessary to supplement with compositions containing only EPA. The apparent transfer efficiency of EPA into milk has been reported by a number of authors to be higher than that of DHA (See Gulatia, S. K et al., International Dairy Journal 13 339-343 (2003); Wright, T. C. et al., J. Dairy Sci. 86: 861-869 American Dairy Science Association (2003.) It has been also been proposed that DHA is retroconverted to EPA in ruminants (See Barclay, W., et al., Dietetics, 83: 61-76 (1998).

Until now however, no in vivo bovine supplementation studies using compositions containing substantially only EPA have been reported in the literature and there is little information if any as to the extent to which EPA may be metabolized to DHA in bovines. We wished to investigate whether supplementing the diets of lactating dairy cows with a composition containing substantially only EPA would lead to the production of EPA-only milk.

### Materials and Methods

Ethical approval was obtained from the University of Auckland Animal Ethics Committee to undertake an pilot supplementation study. The study involved supplementing the diets of four Friesian dairy cows with single daily dose of 40 and 160 of ethyl-eicospaentaenoic acid (>98% purity) for nine days. Cows were divided into a high-to-low dose (HTLD) group (n=2) with animals supplemented with 160 grams of EPA at day one with the dose decreasing by 15 grams each day to reach 40 grams at day nine and a low-to-high dose (LTHD) group (n=2) with animals supplemented with 40 grams on day one the dose increasing by 15 grams each day to reach 160 grams at day nine. Feeding and milk sampling was undertaken between 19 and 29 August 2004 at a dairy farm in West Aucldand under the supervision of a supervising veterinarian with experience in caring for the herd. Supplements were given by drenching gun once daily in the morning subsequent to milking. Prior to supplementation each day duplicate 50 ml samples of milk were collected from each animal and stored in 100 ml polyethylene containers in a temperature controlled minus 20 deg C. freezer within one hour of collection. Milk samples were thawed and fatty acids extracted and prepared for analysis. Milkfat was methylated by dissolving in hexane and reaction with 2 molar KOH methanol. 20 milligrams of fat dissolved in 2 mls in N-Hexane then 20 micro-litres of 2 molar KOH methanol was added left to stand for 3 seconds and vortexed vigorously for 30 seconds. The mixture was then allowed to stand for 6 minutes before being neutralized to methyl red with 2 molar HCl in water before being vortexed again and centrifuged (modified method from agilent website also reported in Terry Knight publications) Knight T. W., et al., New Zealand Journal Of Agricultural Research 47:3,: 287-297 SEP (2004). An aliquot of hexane was then taken and analysed by GCFID 6890 (Agilent Corp) with 105 meter 0.25 mm RTX2330 90% bis cyanoprofile column (Restek) and 0.25 micron film thickness. Fatty acids were identified by retention time in comparison with a 37-component mixture (Suplco). Peak areas were corrected for peak response using theoretical response factors (AOCS reference). Results were reported as area under curve.

A permutation based method of statistical analysis was used to assess FA baseline composition with reference to a reference baseline derived from population of four thousand one hundred NZ Fresian dairy cows. Each fatty acid peak measured in the feeding trial was logged (log base 2), and had the day 0 (baseline) measurement subtracted to report a change from baseline at each time point. Significance of the changes in four-cow and two-cow means was tested by randomly selecting many groups of two or four cows from the herd of 4100 animal reference dataset and comparing the feeding trial results against the mean of logged measurements from the larger reference population. This produced baseline distributions for each group size which were centered at zero to match the baseline centered means from the animals in the feeding trial. Significance thresholds were then adjusted for the number of fatty acid peaks being investigated, and the number of measurements taken.

### Results and Discussion

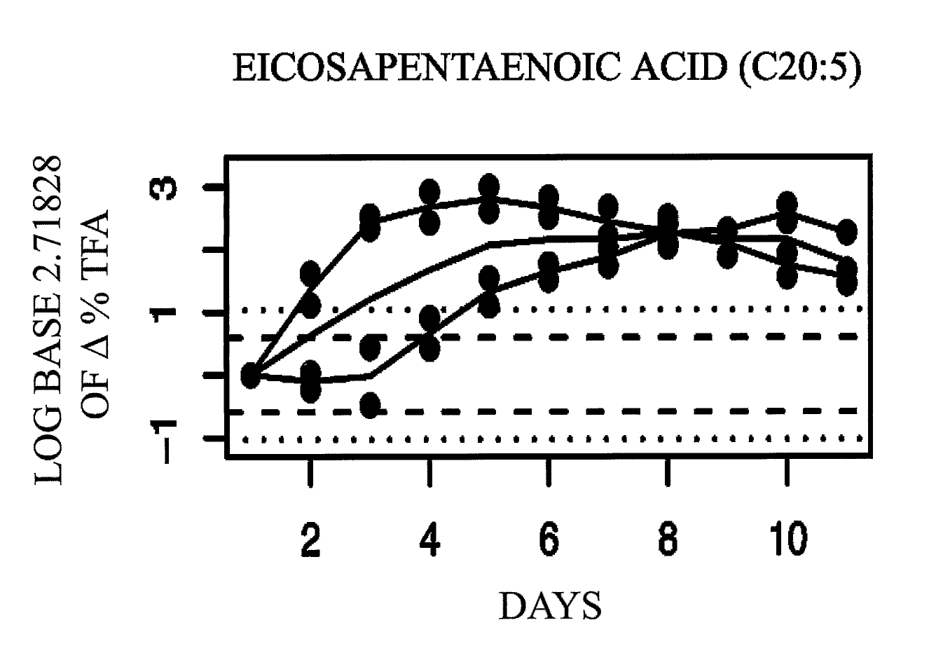

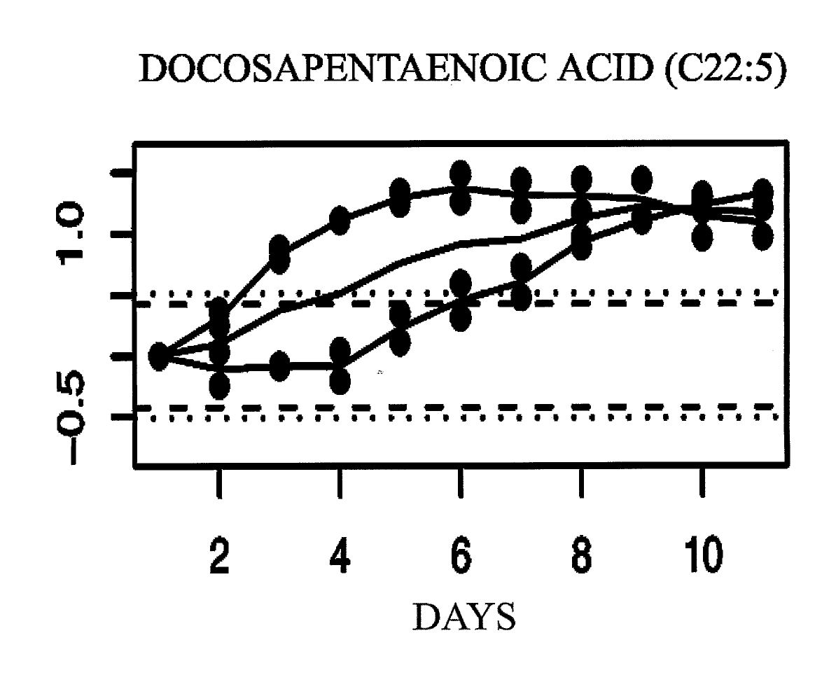

Omega-3 fatty acids. In the present study eicosapentaenoic (C20:5 EPA) and docosapentaenoic acid (C22:5 DPA), rose significantly from baseline in the HTLD group by days two and day three respectively and by days five and seven in LTHD group (See: FIG. 1 and FIG. 2). EPA and DPA as a percentage of total fatty acids (% TFA) peaked at days five (1.55) and six (0.41) respectively in the HTLD group and at days nine (1.29) and eleven (0.38) in the LTHD group. DHA was undetectable at baseline and remained unchanged throughout the study suggesting conversion from EPA to DHA may not take place to any great degree in lactating dairy cows. Previous authors have suggested that metabolic pathways retro-converting DHA to EPA, which occurs in humans under certain dietary conditions, my occur to a greater degree in bovines (Barclay et al, 1998) however Spain et al (1995) reported that duodenally infused fish oil led to higher levels of EPA in plasma than DHA suggesting that DHA may be preferentially sequestered, as it is in humans, into a number of tissues and cellular compartments other than milk (See: Spain J. N., et al., J Dairy Sci 78:1142-1153 (1995)). It can be ruled out that DHA would be released from such compartments and accumulate in milk over longer timescales.

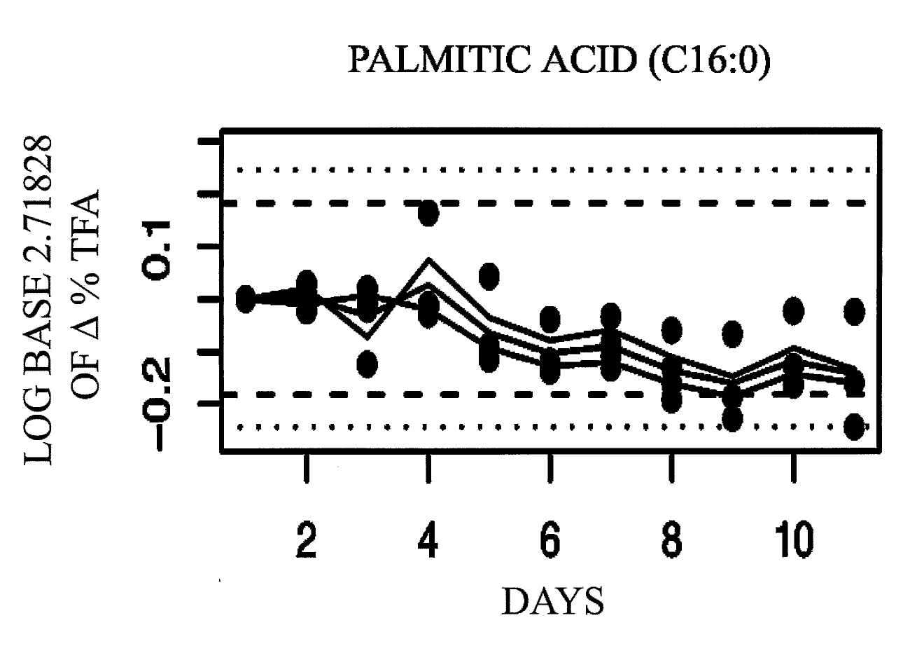

Saturated Fatty Acids: A reduction in dietary amounts of certain saturated fatty acids (SFAs), including in particular the major milk fatty acid C16:0, a has been recommended by health experts (WHO Technical Report Series 916, WHO Geneva (2003)). Under normal circumstances about 50% of dietary C16:0 is taken up by the mammary gland. In the present study, % TFA C16:0 fell consistently throughout the feeding period reaching a maximum reduction of 15.44% of baseline levels at day nine across both groups (31.1 vs. 36.78) (See: FIG. 4) The reduction in C16:0 did not achieve statistical significance when the strict family-wise error rate control method was used due to the very small sample size in the study, but became significant when false discovery rate controlling multiple comparisons and procedures was applied. This decrease in milkfat C16:0 is unlikely to be explained by altered feeding behavior during the study given corresponding changes were not observed in other SFAs. In order to investigate the contribution of further metabolism of C16:0 in the mammary gland to palmitoleic acid (C16:1) we compared substrate/product ratios in milkfat for a number of SFAs and their monounsaturated fatty acid counterparts. These ratios are considered to be a proxy measure for the activity of the delta-9-desaturase activity enzyme. (see Table 1 below).

TABLE 1

| Day/FARatio/% of | | | |
|---|---|---|---|
| baseline. | Baseline | Day Five | Day Eleven |
| C:10:C10:1 | 12.66 (100) | 10.45 (82.5) | 13.46 (106.4) |
| C:14:C14:1 | 14.93 (100) | 12.44 (83.3) | 16.51 (110.6) |
| C:16:C16:1 | 17.40 (100) | 15.76 (90.6) | 19.67 (113.4) |
| C:18:C18:1 | 0.49 (100) | 0.32 (65.3) | 0.48 (98) |

Ratios were significantly diminished in favor of monounsaturates at day five across both groups suggesting an initial upregulation in delta-9-desaturase activity most likely triggered by a dramatic reduction in milkfat stearic acid (C18:0) which was already significant by day three. The reduction in availability of C18:0 to the mammary gland can be explained by an inhibition by EPA of the biohydrogenation by rumen microorganisms of linoleic (LA C18:2) and alpha-linolenic (ALA C18:3) which has been demonstrated by AbuGazelah et al., in vitro (2004) (See AbuGhazaleh AA, et al., J Dairy Sci., Mar; 87:3 645-51 (2004)). As can be see above, ratios bounced back strongly in favor of saturates exceeding baseline levels (with the exception of C18: to C18:1). By day eleven most likely due to the combined effects of an “acclimatization” of rumen microorganisms and a downregulation of delta-9-desaturase in response to the recent increase in the total availability of unsaturated fatty acids to the mammary gland. While desaturation of C16:0 slowed the decrease of C16:0 in milkfat continued indicating other mechanism(s) are responsible for the observed reduction in C16:0 in milkfat. The level of C16:0 in the diet of lactating dairy cows is reflected to a great degree in serum levels. Additionally, it is believed that the uptake of C16:0 from plasma by the mammary gland of cows occurs to a significant degree. Therefore the level of C16:0 in the diet will exert a direct influence on milk composition, Changes in C16:0 levels in milk after dietary change are also under the control of other mechanisms which are not yet understood. Mashek et al (2002) produced a significant increase in oxidation of C16:0 in bovine hepatocytes by supplementing these cells with EPA in vitro (See Aiello, R. J., and L. E. Armentano, Comp. Biochem. Physiol. B. 91:2, 339-344 (1988); Belury M. A., Nutr Rev. 53: 83-89 (1995); Rainer & Heiss, Journal of the American Dietetic Association 104:6, 963-8 (2004); Mashek, D. G. et al., J. Dairy Sci. 85:2283-2289 American Dairy Science Association, (2002)).

To the best of inventors knowledge, neither Mashek et al. nor the previous literature provide any information as to how this effect might be translated into a beneficial effect in humans by altering the diet of dairy cows to produce human food or dietary supplements with enhanced health-giving characteristics. It is proposed in this study that EPA supplementation led to an increase in the rate of oxidation of C16:0 in the liver and other tissues limiting the amount of C16:0 available for uptake into the mammary gland. This suggests impractical way to utilize changes in the that rate at which C16:0 is metabolized to affect eventual milk levels. Conjugated linoleic acid CLA: Supplementation of dairy cows with fish oil is known to lead to an increased levels of C18:1 ctl 1 vaccenic acid (VA) and conjugated linoleic acid (CLA) in cows milk. CLA is believed to have beneficial health effects in humans when ingested, including anticancer and antidiabetogenic properties (See (Belury M. A. Nutr Rev. 53:83-89 (1995); Rainer & Heiss Journal of the American Dietetic Association. 104: 6,:963-8, (2004)). AbuGazaleh & Jenkins (J. Dairy Sci. 87:1047-1050, (2004)) propose that DHA is the component in fish oil that promotes accumulation of vaccenic acid with EPA showing a similar effects to a lesser extent (AbuGazaleh & Jenkins J. Dairy Sci 87: 645-651, (2004)). However neither AbuGazaleh & Jenkins nor the previous literature provide any information. on the divergent effects of EPA, whether or not these may be quantatively superior to those of DHA, on conjugated linoleic acid accumulation in milk or how these might be translated into a beneficial effect in humans. The present study was not designed to investigate the relative efficiency of EPA as compared to DHA in enhancing milkfat CLA. but nevertheless confirms that supplements containing substantially only EPA are able to increase CLA in vivo which rose to a maximum level of 4.2% of total fatty acids, a 375% increase over baseline levels (mean 274%) an effect which could lead to beneficial therapeutic effects associated with ingestion of EPA-only milk products (See Example 6).

Unless protected from ruminal biohydrogenation both DHA and EPA when fed to cows in fish or micro-algae derived supplements are largely (up to 95%) hydrogenated by rumen microorganisms. This effect is lessened in the presence of increasing dietary loads of certain fatty acids possibly including higher levels of EPA over short periods of time, however more economical means of minimising such losses associated are important for the production of milk modified via dietary supplementation of the diets of dairy cows with n-3 HUFAs. Such means could include means to protect against ruminal biohydration and the development of methods and provision of improved supplementation.

It is expected (based on the results of previous studies) that the majority of the test composition was hydrogenated by ruminal microflora. Thus cost effective ruminal protection supplements would be desirable. Although the postulated more efficient pathway for transfer of EPA over and above that for DHA from diet into milk was not studied, the study provides an example of supplementation with EPA and this provides a way, if proven, to enhance the efficiency of the production of EPA as compared to DHA milks.

It has been observed that across a number of studies involving supplementation of the diets of dairy cows with fish based supplements containing both EPA and DHA that EPA was transferred more efficiently into milk than DHA. However the previous literature did not provide any information on how this effect, if it were proven, could be translated into the production of desirable products.

The study did not provide any indication as to the transfer efficiency of EPA into milk however, without substantiating the observed phenomenon of a relatively high transfer efficiency from diet to milk for EPA the study enables the production of EPA-only milk providing for the first time a context in which further work which could leverage the phenomenon.

### EXAMPLE 5

This example describes a method to realise previously unknown beneficial mechanisms and health effects capable of being assessed within a population of animals or of humans, wherein the beneficial mechanisms and health effects are obtained by the production and use in dietary form of compositions produced via heterotrophic or primarily heterotrophic production of micro-algae comprising substantially only EPA as their n-3 HUFA component and relatively low levels of C14:0 and C16:0.

This method involves a) producing a composition; b) feeding the composition to a population of animals or of humans, in a sufficient quantity and over period of time such that beneficial health effects may follow; and c) assessing the beneficial health effects and the mechanisms by which these are obtained. Supplementation may proceed for as little as one day or as long as several years.

A preferred example is a provision of the whole cell composition provided in Example 1 via heterotrophic or largely heterotrophic production of the microalga Nitzschia laevis or an oil extracted therefrom. The whole cell biomass can be provided to a population of animals (n=>1) in a quantity of 0.01 to 10 kilograms a day or an oil extract can be provided to one or more individuals comprising a population of humans (n=>1) in a quantity of 0.1 to 100 grams a day.

Intake of other nutrients can be controlled for by restricting supplying an obligatory diet in the case of farm animals and/or monitoring intake if necessary. In the case of humans this may be achieved by undertaking supplementation whilst humans are housed in a residential metabolic unit so as to attempt to eliminate potential bias generated by uncontrolled dietary intake from a study.

Blood samples or samples of other tissue such as milk or muscle (or eggs, if birds are under study) may be taken from individuals before and after supplementation. Changes in the presence and levels of fatty acids and other beneficial or detrimental nutrients or biochemical markers of disease risk can then be measured in plasma and red blood cells.

Measurements of fatty acids and other biochemical markers of disease risk may then be analyzed to assess the likely health effects of supplementation and mechanisms involved. Assessment methods include (without limitation) fatty acid analysis of tissue FA composition by Gas Chromatography, extraction of nucleic acids, amplification of DNA by polymerase chain reaction, immunohistochemistry protein staining, and other methods.

Health effects include reduction in the clinical signs or symptoms of a disorder including sequelae.

Mechanisms might include transfer of nutrient in composition from diet into tissue, the effect of the composition on other metabolic pathways such as peroxidation of C16:0 in hepatocytes, downregulation of nuclear transcription factors (PPAR gamma etc) substrate availability or chemical type interactions such as dislodgement of other fatty acids from reservoirs.

The invention is intended to satisfy an unsatisfied need by the human population for appropriate intakes/levels of the “essential fatty acids” in particular EPA and DHA. Medical publications are currently uncovering further aspects of the position of the EFAs in health. There is little prospect that the identified world shortfall which could be of the order of a millions kilograms per day can be remedied by reliance on products made from fish caught from the wild. Furthermore it is uneconomical to purify individual n-3 HUFAs from complex mixtures of FAs to satisfy general dietary requirements. The supply of specific n-3 HUFAs at proper levels will enhance the level of health of the population. At this time, no known manufactures appear to be capable of meeting demands. The invention is capable of providing alternative sources of EFAs in the quantities and with the qualities desired for the human population.

The invention provides relatively inexpensive yet relatively pure dietary or nutritional supplements, or even ordinary foods of known types (such as milk and butter having enhanced health characteristics obtained indirectly from food-producing animals), wherein the provided materials offer a relatively uncontaminated, inexpensive, and acceptable source of a specified n-3 HUFA. Most versions of the products will have the effect of boosting levels of selected EFAs and other beneficial fatty acids in a human lipid profile while at the same time reducing the health risks posed by dietary intake of C14:0 and C16:0 and other non-beneficial or harmful EFAs.

The cost effectiveness and general acceptability depends in part on selection of an appropriate micro-algal biomass as a starting material, together with minimized need for post-harvest processing such as steps that alter the lipid profile of the material as harvested.

### EXAMPLE 6

### Human Consumption of Enhanced Epa Containing Milk

This example describes the incorporation of n-3 HUFAs into into human tissue via consumption of EPA-only dairy products. In a preferred embodiment the method comprised (i) producing an EPA-only milk via supplementation of lactating dairy cows with an EPA only supplement relatively low levels of myristic and plamitic acids, (ii) harvesting the milk from said cows, (iii), processing said milk into other dairy products (including creamy milk and enriched creams,) suitable for consumption by a human. (iv) these were then consumed by a human over a sufficiently lengthy period of time such that sufficient n-3 HUFA component of the said dairy products were absorbed by the human and incorporated into tissue.

A 38 year old healthy male subject was fed creamy milk and enriched creams prepared from milk produced via the method of Example 3 over a period two days in an attempt to demonstrate absorption and incorporation of fatty acids from the said dairy products into blood serum and red blood cells.

Preparation of Enriched Foodstuffs.

One creamy milk (Creamy Milk) and two enriched creams (Enriched Cream A and Enriched Cream B) were prepared. The Creamy Milk was prepared by allowing milk to settle in three 20 liter containers overnight at 4 deg C. and then drawing creamy milks off the top of the containers and homogenizing the combined creamy milks. The two Enriched Creams were further prepared by centrifuging said Creamy Milk in 600 ml centrifuge bottles for 5-minutes at 2000 g ,scooping the enriched cream from the top of the bottles, and then homogenizing sadi Enrichedf Creams. Creamy milk and Enriched Creams were then incorporated into a variety of n-3 foodstuffs including fruit smoothies produced by liquidizing bananas and strawberries with creamy milk and enriched cream, creamy beef stew produced by mixing enriched cream into an existing beef stew, chocolate log cake with whipped enriched cream prepared by whipping the enriched cream and placing on top of an existing chocolate log cake. These n-3HUFA enriched food stuffs were then made available for consumption.

Milk-Fat Analysis.

Samples from creamy milk and enriched creams were stored frozen for 2-3 days at 20 deg C. and then thawed for fatty acid extraction and analysis by gas chromatography Fatty acid samples were prepared for analysis by adding approximately 30 mg (20 uL) of melted fat (60° C.) to a 15 mL kimmax tube and dissolving in 2 ml of heptane and the adding 20 uL of 2M KOH in methanol. The samples were then vortexed for 30 sec until cloudy and allowed to stand for 5 mins. The mixtures were then neutralized by adding 25 uL of 2M HCL to produce a red color, vortexed again until well mixed and then centrifuged at 3,000 rpm for 2 mins. The upper heptane layer of the samples was then removed with a Pasteur pipette and placed in an auto-sampler vial for Gas Chromatography (GC) analysis. GC analysis was undertaken in a Gas Chromatograph fitted with a flame ionization detector (GCFID 6890; Agilent Corp) with 105 meter 0.25 mm 90% bis cyanoprofile column (RTX2330; Restek) with a 0.25 micron film thickness. Fatty acids were identified by retention time in comparison with a 37-component mixture (Suplco). Peak areas were corrected for peak response using theoretical response factors (AOCS reference) and results reported as area under the curve.

Blood Analysis.

Baseline post-prandial (4 hours after a normal mid-day meal) and fasting (after 10 hours of fasting) blood samples were taken prior to supplementation commencing and at 24 hours and 39 hours respectively after supplementation had commenced. Serum samples were prepared by allowing blood to congeal for one hour and then centrifuging the blood at 1000 g for 15 minutes and drawing serum off the top of the centrifuge tube. Red blood cells (RBC) were prepared by centrifuging at 1000 g for two minutes, drawing off serum and then repeating the following method twice; washing the cells with phosphate-buffered physiological saline solution adjusted to pH 7.4 prior to centrifuging again and drawing off fluid. Serum and RBC samples were frozen for 2-3 days at 20 deg C. and then thawed for fatty acid extraction and analysis by gas chromatography.

Milkfat Analysis

Lipid analysis indicated that creamy milk contained 6.70% total lipid by weight and that enriched creams 1 and 2 contained 53.74% and 49.85% total lipid by weight respectively. N-3 HUFA content as a percentage of total milk fatty acids was 0.49, 0.57 and 0.41% for EPA, 0.12, 0.12, and 0.11% for DPA and 0.04, 0.04 and 0.03% DHA for creamy milk, and enriched creams 1 and 2 respectively. C16:0 comprised 21.84, 21.80 and 22.04% of total fatty acids and CLA comprised 2.06, 2.21 and 2.17% in creamy milk, and enriched creams 1 and 2 respectively. It was presumed, as is known in the art, that 80% of the lipid was comprised of fatty acids.

Consumption of n-3 HUFA Enriched Foods.

The 38 year old subject had been a regular user of purified fish oil capsules but had ceased taking supplements two weeks prior to the start of the demonstration. During the course of the demonstration the subject consumed no fish and no fish oil or omega-3 supplements. With the exception of the creamy milk and enriched creams the remainder of the supplemented diet consumed was also relatively low in alpha-linolenic acid (C18:3 n-3) comprising no leafy green vegetables, flax seed oil or other rich sources of omega-3 fatty acids. In the first 24 hours of supplementation enriched foodstuffs containing 3 kilograms of creamy milk, 250 grams of enriched cream 1 and 400 grams enriched cream 2 were consumed. Between 24 and 30 hours an additional 2.0 kilograms of creamy milk and an additional 400 grams of enriched cream 2 were consumed in enriched foodstuffs.

From this it can be calculated that in the first 24 hours post supplementation 160.8 grams of milk fatty acids were consumed creamy milk 107.5 from grams of fatty acids from enriched cream 1 and 159.52 grams of fatty acids from enriched cream 2 totaling 427.8 grams of milk fatty acids. In the last 5-6 hours of supplementation an additional 107.2 grams of milk fatty acids were consumed from creamy milk and an additional 159.52 grams of fatty acids from enriched cream 2 totaling 266.72 grams of milk fatty acids. This equates in the first 24 hours of supplementation to intakes for EPA, DPA and DHA of 2.1, 0.5 and 0.2 grams and in the last 5-6 hours of 1.3, 0.3 and 0.1 grams respectively. C16:0 and CLA intakes can be calculated as 93.6 and 9.2 grams in the first 24 hours and 34.9 and 3.42 grams in the last 5-6 hours of supplementation.

Fatty Acids in Blood Samples.

At baseline the EPA and DPA and DHA as a percentage of total fatty acids in fasting serum samples was 1.36, 0.84 and 3.0 respectively as compared to 3.74, 0.97 and 2.5 in serum from blood taken 39 hours post supplementation. In post-prandial serum samples EPA and DPA and DHA were measured as 1.44, 0.90, 3.50 percent of total fatty acids at baseline respectively versus of 2.23, 0.72, and 1.89 percent in samples taken 24 hours post supplementation. In post-prandial red blood cells EPA and DPA and DHA were measured as 0.92 1.98 and 6.9 percentage of total fatty acids respectively at baseline versus 1.55, 3.01 and 8.39 respectively in samples taken 24 hours post-supplementation. CLA levels in blood were not measured.

C16:0 in fasting serum was measured at 23.11 percent of total fatty acids at baseline versus 20.91 thirty-nine hours post-supplementation, and in post-prandial serum fatty acids at 21.8 at baseline versus 22.72 twenty-four hours post-supplementation. C16:0 in post-prandial red blood cell fatty acids fell from 22.22 at baseline to 19.74 twenty four hours post supplementation. C14:0 was measured in baseline fasting serum at 2.38% of total fatty acids and fell at 39 hours post supplementation to 2.23%. C14:0 however rose in post prandial serum and red blood cells from 1.42% at baseline to 4.71% at 24 hours post-supplementation in serum and from 0.43% to 0.51% in red blood cells.

This is the first known example of a milk containing significant levels of EPA but substantially no DHA being fed to humans. The 2.75-fold increase measured in the proportion of EPA in fasting serum fatty acids demonstrates that EPA was absorbed well in a human after consumption of the EPA only dairy products. This conclusion is supported by the more than 1.5-fold increase in the proportion of this highly unsaturated omega-3 fatty acid measured in post-prandial serum fatty acids. The measured decrease in serum DHA over the period of supplementation may be explained by the displacement of DHA from serum triglycerides making these available for absorption into tissue. This explanation is consistent with the observed marked rise in the proportion of fatty acids accounted for by DHA in red blood cells prepared from post-prandial blood samples taken at baseline versus 24 hours post supplementation (6.9 versus 8.39). Whilst significant DPA was supplied in the supplement it is interesting to note that the proportion of DPA in serum samples did not change appreciably. The proportion of fatty acids in post-prandial red blood samples accounted for by DPA rose significantly from baseline compared to 24 hours post-supplementation (1.98 versus 3.01). Clearly consumption on a regular basis of th quantity of saturated fatty acids and in particular C16:0 which were consumed over this period of time is unadvisable. Perhaps surprisingly, the most obvious change observed in C16:0 was the measured decrease from 22.22 to 19.74% of total fatty acids in postprandial red blood cells from baseline as compared to 24 hours post-supplementation wheras C14:0 appeared to increase slightly from 0.43 to 0.51%. It is conceivable that the increased levels of beneficial fatty acids in the EPA Milk increased oxidation of this saturated fatty acid although the data is inconclusive due to a number of factors including the short duration of the trial, the small sample size and the difficulty of controlling for confounds in the form of the contribution of common dietary fatty acids from other sources at baseline.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1-72. (canceled)

73. A method of producing a composition comprising by weight of total fatty acids:
i. between 10% and about 50% n-3 HUFA;
ii. less than about 9% myristic acid (C14:0);
iii. less than about 20% palmitic acid (C16:0), and
iv. a ratio of EPA to DHA in the composition of at least 7:1, and wherein said composition is produced by a primarily heterotrophic culture.

74. The method of claim 73 wherein the composition is produced in a hybrid of a fermentor and a photobioreactor.

75. The method of claim 73 wherein the specified ratio of EPA to DHA and amounts of EPA, myristic acid, and palmitic acid in the composition are obtained without recourse to further purification or dilution after growth of the culture is terminated.

76. The method of claim 73 wherein the specified ratio of EPA to DHA and amounts of EPA, myristic acid, and palmitic acid in the composition are obtained with recourse to further purification or dilution after growth of the culture is terminated.

77. The method of claim 73 wherein the composition is produced by a commercially cultivated microorganism.

78. The method of claim 73 wherein the composition is produced by in vitro culture of naturally occurring or transgenic microorganisms; the genes of which organisms comprise micro-algal genes.

79. The method of claim 73 wherein the composition is produced by primarily heterotrophic culture of the micro-algal species Nitzschia laevis.

80. A composition comprising by weight of total fatty acids 5 to 50% EPA;
less than about 16% palmitic acid (C16:0);
less than 8% myristic acid (C14:0), and
wherein the ratio of EPA to DHA in the composition of at least 7:1.

81. A composition comprising by weight of total fatty acids 10 to 50% n-3 HUFA;
less than about 20% palmitic acid (C 16:0);
less than 7% myristic acid (C14:0), and
wherein the ratio of EPA to DHA in the composition is at least 7:1.

82. The method of claim 73 wherein the ratio of EPA to DHA in the composition is at least 9:1.

83. The composition of claim 80 wherein the ratio of EPA to DHA in the composition is at least 9:1.

84. A biomass comprising cells of a microorganism produced in an in vitro culture; the biomass having a total fatty acid composition as claimed in claim 80.

85. A nutritional supplement for human consumption which comprises a composition produced by the method of claim 73.

86. The composition of claim 80 formulated as a nutritional supplement for human consumption.

87. A nutritional supplement for consumption by an animal which comprises a composition produced by the method of claim 73.

88. The composition of claim 80 formulated as a nutritional supplement for consumption by an animal.

89. A nutritional supplement in a form selected from the group consisting of food ingredients, functional foods, dietary supplements and nutraceuticals which comprises a composition produced by the method of claim 73.

90. The composition of claim 80 when used in a form selected from the group consisting of food ingredients, functional foods, dietary supplements and nutraceuticals.

91. A food product for human consumption which comprises a composition produced by the method of claim 73.

92. The composition of claim 80 formulated as a food product for human consumption.

93. A nutritional supplement formulated as a food product for human consumption which comprises a composition produced by the method of claim 73.

94. A growing up formula suitable for consumption by children over one year of age which comprises a composition produced by the method of claim 73.

95. The composition of claim 80 formulated as a growing up formula suitable for consumption by children over one year of age.

96. A geriatric formula suitable for consumption by the elderly which comprises a composition produced by the method of claim 73.

97. The composition of claim 80 formulated as a geriatric formula suitable for consumption by the elderly.

98. A capsule, pill, or dosage form suitable for oral administration to a human which comprises a composition produced according to the method of claim 73.

99. The composition of claim 80 formulated as a capsule, pill, or dosage form for oral administration to a human.

100. A method of increasing levels of EPA in serum and red blood cells in an animal over time with no significant concomitant increase in myristic acid (C14.0) or palmitic acid (C16.0) which comprises administering to the animal an effective amount of a composition produced according to the method of claim 73.

101. A method of increasing levels of EPA in serum and in red blood cells in an animal over time with no significant concomitant increase in myristic acid (C14.0) or palmitic acid (C16.0) which comprises administering to the animal an effective amount of the composition of claim 80.

102. A method of increasing levels of EPA in serum and in red blood cells in a human over time with no significant concomitant increase in myristic acid (C14.0) or palmitic acid (C16.0) which comprises administering to the human an effective amount of a composition produced according to the method of claim 73.

103. A method of increasing levels of EPA in serum and in red blood cells in a human over time with no significant concomitant increase in myristic acid (C14.0) or palmitic acid (C16.0) which comprises administering to the human an effective amount of the composition of claim 80.

104. A method of treatment of a disorder in a patient, which disorder is selected from the group consisting of complications of diabetes (type I, and type II), diabetes-associated hypertension, cancer, osteoarthritis, inflammatory and auto-immune diseases, respiratory diseases, neurological disorders, neurodegenerative disorders, renal and urinary tract disorders, cardiovascular disorders, cerebrovascular disorders, degenerative diseases of the eye, depression, psychiatric disorders, reproductive disorders, visceral disorders, muscular disorders, metabolic disorders, prostatic hypertrophy and prostatitis, impotence and male infertility, mastalgia, male pattern baldness, osteoporosis, dermatological disorders, dyslexia and other learning disabilities, and cancer cachexia, obesity, ulcerative colitis, Crohn's disease, anorexia nervosa, burns, osteoarthritis, osteoporosis, attention deficit/hyperactivity disorder, and early stages of colorectal cancer, lung and kidney diseases, and disorders associated with abnormal growth and development comprising administering to a patient an effective amount of a composition produced by the method of claim 73.

105. A method of treatment of a disorder in a patient, which disorder is selected from the group consisting of complications of diabetes (type I, and type II), diabetes-associated hypertension, cancer, osteoarthritis, inflammatory and auto-immune diseases, respiratory diseases, neurological disorders, neurodegenerative disorders, renal and urinary tract disorders, cardiovascular disorders, cerebrovascular disorders, degenerative diseases of the eye, depression, psychiatric disorders, reproductive disorders, visceral disorders, muscular disorders, metabolic disorders, prostatic hypertrophy and prostatitis, impotence and male infertility, mastalgia, male pattern baldness, osteoporosis, dermatological disorders, dyslexia and other learning disabilities, and cancer cachexia, obesity, ulcerative colitis, Crohn's disease, anorexia nervosa, burns, osteoarthritis, osteoporosis, attention deficit/hyperactivity disorder, and early stages of colorectal cancer, lung and kidney diseases, and disorders associated with abnormal growth and development comprising administering to a patient an effective amount of a medicament comprising the composition of claim 80.

106. A method of making an animal food product which comprises the step of administering or providing a composition produced according the method of claim 73 to an animal in an amount and for a time sufficient to alter the lipid composition in a selected tissue or fluid of said animal, and harvesting a selected animal tissue or fluid for use as a food product.

107. A method of making an animal food product which comprises the method comprising the step of administering or providing the composition of claim 80 to an animal in an amount and for a time sufficient to alter the lipid composition in a selected tissue or fluid of said animal, and harvesting a selected animal tissue or fluid for use as a food product.

108. A method of making an animal food product which comprises the step of administering or providing a composition comprising substantially only EPA in its n-3 HUFA fraction to an animal in an amount and for a time sufficient to alter the lipid composition in a selected tissue or fluid of said animal, and harvesting a selected animal tissue or fluid for use as a food product.

109. A milk based food product made according to the method of claim 108.

110. A milk-based food product made according to the method of claim 106 selected from the group consisting of butter, cheese, chocolate, cottage cheese, cream, milk, powdered milk, condensed milk, skim milk, ice cream, yogurt and infant-formula.

111. A fluid harvested according to claim 108 comprising at least about 0.4% EPA by weight of total fatty acids; and a ratio of EPA to DHA of more than 5:1.

112. A method of increasing levels of EPA in serum and in red blood cells in a human over time with no significant concomitant increase in myristic acid (C14.0) or palmitic acid (C16.0) which comprises administering to the human an effective amount of a composition produced according to the method of claim 108.

113. A method of treatment of a disorder in a patient, which disorder is selected from the group consisting of complications of diabetes (type I, and type II), diabetes-associated hypertension, cancer, osteoarthritis, inflammatory and auto-immune diseases, respiratory diseases, neurological disorders, neurodegenerative disorders, renal and urinary tract disorders, cardiovascular disorders, cerebrovascular disorders, degenerative diseases of the eye, depression, psychiatric disorders, reproductive disorders, visceral disorders, muscular disorders, metabolic disorders, prostatic hypertrophy and prostatitis, impotence and male infertility, mastalgia, male pattern baldness, osteoporosis, dermatological disorders, dyslexia and other learning disabilities, and cancer cachexia, obesity, ulcerative colitis, Crohn's disease, anorexia nervosa, burns, osteoarthritis, osteoporosis, attention deficit/hyperactivity disorder, and early stages of colorectal cancer, lung and kidney diseases, and disorders associated with abnormal growth and development comprising administering to a patient an effective amount of a composition produced by the method of claim 108.
